(19) United States
(12) Patent Application Publication
(10) Pub. No.: US 2005/0084850 A1
(43) Pub. Date: Apr. 21, 2005

(54) Bone marrow secreted proteins and polynucleotides

(76) Inventors: Li Cao, St. Louis, MO (US); Haishan Lin, Castro Valley, CA (US)

Correspondence Address: Chiron Corporation;Intellectual Property, R338, P.O. Box 8097, Emeryville, CA, 94662-8097, US

(21) Appl. No.: 10/347,669

(22) Filed: Jan. 16, 2003

Related U.S. Application Data
(63) Continuation of application No. 09/765,205, filed on Jan. 17, 2001, now abandoned.
Application No. 09/765,205 is a division of application No. 09/212,440, filed on Dec. 16, 1998, now abandoned.
(60) Provisional application No. 60/068,958, filed on Dec. 30, 1997.
(60) Provisional application No. 60/101,603, filed on Sep. 24, 1998.
(60) Provisional application No. 60/102,540, filed on Sep. 30, 1998.

(51) Int. Cl. C12Q 1/68; C07H 21/04; C12N 9/64

(52) U.S. Cl. 435/6; 435/69.1; 435/320.1; 435/325; 435/226; 530/350; 536/23.2

(57) ABSTRACT

Novel polynucleotides and secreted proteins encoded thereby are disclosed. The proteins can be used as therapeutics, for example, to stimulate blood cell generation in patients receiving cancer chemotherapy, to treat bone marrow transplantation patients, and to heal fractured bones. Polynucleotides of the invention can be used therapeutically, to provide proteins of the invention. Polynucleotides of the invention can also be used diagnostically, such as on polynucleotide arrays, to detect differential gene expression in diseased tissue compared with gene expression in normal tissue.

This application claims the benefit of co-pending provisional applications Ser. No. 60/068,958, filed Dec. 30, 1997, Ser. No. 60/101,603, filed Sep. 24, 1998, and Ser. No. 60/102,540 filed Sep. 30, 1998, which are incorporated herein by reference.

### TECHNICAL AREA OF THE INVENTION

This invention relates to proteins secreted from bone marrow and to polynucleotides encoding the -secreted proteins. The invention also relates to therapeutic and diagnostic utilities for the polynucleotides and proteins.

### BACKGROUND OF THE INVENTION

Bone marrow stromal cells secrete a variety of protein factors required for the formation of blood and bone cells and for other physiological processes. Known regulatory factors involved in hematopoiesis and/or bone development include SCF, IL3, IL-6, GM-CSF, M-CSF, EPO, TPO, bone morphogenic proteins, erythroid potentiating factor, and TGF-β. However, it is believed that additional secreted protein factors which control hematopoiesis and bone morphogenesis remain to be identified.

### SUMMARY OF THE INVENTION

It is an object of the invention to provide proteins secreted from bone 20 marrow stromal cells and polynucleotides encoding the secreted proteins. These and other objects of the invention are provided by one or more of the embodiments described below.

One embodiment of the invention is an isolated and purified protein comprising an amino acid sequence which is at least 85% identical to an amino acid sequence selected from the group consisting of SEQ ID NOS:2, 4, 6, 8, 10, 12, 14, 16, 18, 25 20, 22, 24, 26, 28, 30, 32, 34, 36, 38, 40, 42, and 44. Percent identity is determined using a Smith-Waterman homology search algorithm using an affine gap search with a gap open penalty of 12 and a gap extension penalty of 1.

Another embodiment of the invention is an isolated and purified protein comprising an amino acid sequence selected from the group consisting of at least 95 contiguous amino acids of SEQ ID NO:2, at least 101 contiguous amino acids of SEQ ID NO:4, at least 14 contiguous amino acids selected from amino acids 1-312 of SEQ ID NO:6, at least 179 contiguous amino acids of SEQ ID NO:6, at least 75 contiguous amino acids of SEQ ID NO:6, at least 179 contiguous amino acids of SEQ ID NO:8, at least 136 contiguous amino acids of SEQ ID NO:8, at least 17 contiguous amino acids selected from amino acids 1-287 of SEQ ID NO:8, at least 82 contiguous amino acids of SEQ ID NO:10, at least 31 contiguous amino acids selected from amino acids 1-238 of SEQ ID NO:10, at least 96 contiguous amino acids of SEQ ID NO:12, at least 27 contiguous amino acids selected from amino acids 250-383 of SEQ ID NO:12, at least 6 contiguous amino acids selected from amino acids 1-184 of SEQ ID NO:12, at least 8 contiguous amino acids selected from amino acids 268-364 of SEQ ID NO:12, at least 104 contiguous amino acids of SEQ ID NO:14, at least 75 contiguous amino acids of SEQ ID NO:14, at least 17 contiguous amino acids selected from amino acids 1-150 of SEQ ID NO:14, at least 6 contiguous amino acids selected from amino acids 204-261 of SEQ ID NO:14, at least 6 contiguous amino acids selected from amino acids 1-111 of SEQ ID NO:14; at least 8 contiguous amino acids of SEQ ID NO:16, at least 46 contiguous amino acids of SEQ ID NO:18, at least 3.9 contiguous amino acids selected from amino acids 13-232 of SEQ ID NO:18; at least 6 contiguous amino acids of SEQ ID NO:20, at least 7 contiguous amino acids of SEQ ID NO:22, at least 7 contiguous amino acids of SEQ ID NO:24, at least 11 contiguous amino acids of SEQ ID NO:26, at least 257 contiguous amino acids of SEQ ID NO:28, at least 6 contiguous amino acids selected from amino acids 1-31 of SEQ ID NO:28, at least 6 contiguous amino acids of SEQ ID NO:30, at least 117 contiguous amino acids of SEQ ID NO:32, at least 6 contiguous amino acids selected from amino acids 1-65 of SEQ ID NO:32, at least 6 contiguous amino acids of SEQ ID NO:34, at least 14 contiguous amino acids of SEQ ID NO:36, at least 19 contiguous amino acids of SEQ ID NO:38, at least 8 contiguous amino acids of SEQ ID NO:40, at least 7 contiguous amino acids of SEQ ID NO:42, and at least 10 contiguous amino acids of SEQ ID NO:44.

Still another embodiment of the invention is a fusion protein comprising two protein segments joined together with a peptide bond. The first protein segment consists of an amino acid sequence selected from the group consisting of at least 95 contiguous amino acids of SEQ ID NO:2, at least 101 contiguous amino acids of SEQ ID NO:4, at least 14 contiguous amino acids selected from amino acids 1-312 of SEQ ID NO:6, at least 179 contiguous amino acids of SEQ ID NO:6, at least 75 contiguous amino acids of SEQ ID NO:6, at least 179 contiguous amino acids of SEQ ID NO:8, at least 136 contiguous amino acids of SEQ ID NO:8, at least 17 contiguous amino acids selected from amino acids 1-287 of SEQ ID NO:8, at least 82 contiguous amino acids of SEQ ID NO:10, at least 31 contiguous amino acids selected from amino acids 1-238 of SEQ ID NO:10, at least 96 contiguous amino acids of SEQ ID NO:12, at least 27 contiguous amino acids selected from amino acids 250-383 of SEQ ID NO:12, at least 6 contiguous amino acids selected from amino acids 1-184 of SEQ ID NO:12, at least 8 contiguous amino acids selected from amino acids 268-364 of SEQ ID NO:12, at least 104 contiguous amino acids of SEQ ID NO: 14, at least 75 contiguous amino acids of SEQ i) NO: 14, at least 17 contiguous amino acids selected from amino acids 1-150 of SEQ ID NO:14, at least 6 contiguous amino acids selected from amino acids 204-261 of SEQ ID NO:14, at least 6 contiguous amino acids selected from amino acids 1-111 of SEQ ID NO:14, at least 8 contiguous amino acids of SEQ ID NO:16, at least 46 contiguous amino acids of SEQ ID NO:18, at least 39 contiguous amino acids selected from amino acids 13-232 of SEQ ID NO:18; at least 6 contiguous amino acids of SEQ ID NO:20, at least 7 contiguous amino acids of SEQ ID NO:22, at least 7 contiguous amino acids of SEQ ID NO:24, at least 11 contiguous amino acids of SEQ ID NO:26, at least 257 contiguous amino acids of SEQ ID NO:28; at least 6 contiguous amino acids selected from amino acids 1-31 of SEQ ID NO:28, at least 6 contiguous amino acids of SEQ ID NO:30, at least 117 contiguous amino acids of SEQ ID NO:32, at least 6 contiguous amino acids selected from amino acids 1-65 of SEQ ID NO:32, at least 6 contiguous amino acids of SEQ ID NO:34, at least 14 contiguous amino acids of SEQ ID NO:36, at least 19 contiguous amino acids of SEQ ID NO:38, at least 8 contiguous amino acids of SEQ ID NO:40, at least 7 contiguous amino acids of SEQ ID NO:42, and at least 10 contiguous amino acids of SEQ ID NO:44.

Even another embodiment of the invention is a preparation of antibodies which specifically binds to a protein comprising an amino acid sequence selected from the group consisting of SEQ ID NOS:2, 4, 6, 8, 10, 12, 14, 16, 18, 20, 22, 24, 26, 28, 30, 32, 34, 36, 38, 40, 42, and 44.

Still another embodiment of the invention is an isolated and purified subgenomic polynucleotide which encodes a protein comprising an amino acid sequence which is at least 85% identical to an amino acid sequence selected from the group consisting of SEQ ID NOS:2, 4, 6, 8, 10, 12, 14, 16, 18, 20, 22, 24, 26, 28, 30, 32, 34, 36, 38, 40, 42, and 44. Percent identity is determined using a Smith-Waterman homology search algorithm using an affine gap search with a gap open penalty of 12 and a gap extension penalty of 1.

A further embodiment of the invention is an isolated and purified subgenomic polynucleotide comprising a nucleotide sequence which is at least 85% identical to a nucleotide sequence selected from the group consisting of SEQ ID NOS:1, 3, 5, 7, 9, 11, 13, 15, 17, 19, 21, 23, 25; 27, 31, 33, 35, 37, 39, 41, 43, 45, and the complements thereof. Percent identity is determined using a Smith-Waterman homology search algorithm using an affine gap search with a gap open penalty of 12 and a gap extension penalty of 1.

Another embodiment of the invention is an isolated and purified subgenomic polynucleotide which encodes an amino acid sequence selected from the group consisting of at least 95 contiguous amino acids of SEQ ID NO:2; at least 101 contiguous amino acids of SEQ ID NO:4, at least 14 contiguous amino acids selected from amino acids 1-312 of SEQ ID NO:6; at least 179 contiguous amino acids of SEQ ID NO:6, at least 75 contiguous amino acids of SEQ ID NO:6, at least 179 contiguous amino acids of SEQ ID NO:8, at least 136 contiguous amino acids of SEQ ID NO:8, at least 17 contiguous amino acids selected from amino acids 1-287 of SEQ ID NO:8, at least 82 contiguous amino acids of SEQ ID NO:10, at least 31 contiguous amino acids selected from amino acids 1-238 of SEQ ID NO:10, at least 96 contiguous amino acids of SEQ ID NO:12, at least 27 contiguous amino acids selected from amino acids 250-383 of SEQ ID NO:12, at least 6 contiguous amino acids selected from amino acids 1-184 of SEQ ID NO: 12, at least 8 contiguous amino acids selected from amino acids 268-364 of SEQ ID NO:12, at least 104 contiguous amino acids of SEQ ID NO:14, at least 75 contiguous amino acids of SEQ ID NO:14, at least 17 contiguous amino acids selected from amino acids 1-150 of SEQ ID NO:14, at least 6 contiguous amino acids selected from amino acids 204-261 of SEQ ID NO:14, at least 6 contiguous amino acids selected from amino acids 1-111 of SEQ ID NO:14, at least 8 contiguous amino acids of SEQ ID NO:16, at least 46 contiguous amino acids of SEQ ID NO:18, at least 39 contiguous amino acids selected from amino acids 13-232 of SEQ ID NO:18, at least 6 contiguous amino acids of SEQ ID NO:20, at least 7 contiguous amino acids of SEQ ID NO:22, at least 7 contiguous amino acids of SEQ ID NO:24, at least 11 contiguous amino acids of SEQ ID NO:26, at least 257 contiguous amino acids of SEQ ID NO:28; at least 6 contiguous amino acids selected from amino acids 1-31 of SEQ ID NO:28, at least 6 contiguous amino acids of SEQ ID NO:30, at least 117 contiguous amino acids of SEQ ID NO:32, at least 6 contiguous amino acids selected from amino acids 1-65 of SEQ ID NO:32, at least 6 contiguous amino acids of SEQ ID NO:34, at least 14 contiguous amino acids of SEQ ID NO:36, at least 19 contiguous amino acids of SEQ ID NO:38, at least 8 contiguous amino acids of SEQ ID NO:40, at least 7 contiguous amino acids of SEQ ID NO:42, and at least 10 contiguous amino acids of SEQ ID NO:44.

Still another embodiment of the invention is an isolated and purified subgenomic polynucleotide comprising a polynucleotide segment which hybridizes to a nucleotide sequence selected from the group consisting of SEQ ID NOS:1, 3, 5, 7, 9, 11, 13, 15, 17, 19, 21, 23, 25, 27, 31, 33, 35, 37, 39, 41, and 43, and the complements thereof after washing with 0.2×SSC at 65° C., wherein the polynucleotide segment encodes a protein having an amino acid sequence selected from the group consisting of SEQ ID NOS:2, 4, 6, 8, 10, 12, 14, 16, 18, 20, 22, 24, 26, 28, 30, 32, 34, 36, 38, 40, 42, and 44.

Even another embodiment of the invention is an isolated and purified subgenomic polynucleotide comprising a nucleotide sequence selected from the group consisting of at least 499 contiguous nucleotides of SEQ ID NO: 1, at least 1141 contiguous nucleotides of SEQ ID NO:1, at least 475 contiguous nucleotides of SEQ ID NO:3, at least 313 contiguous nucleotides selected from nucleotides 1-1001 of SEQ ID NO:3, at least 751 contiguous nucleotides of SEQ ID NO:5, at least 538 contiguous nucleotides of SEQ ID NO:5, at least 11 contiguous nucleotides selected from nucleotides 1-946 of SEQ ID NO:5, at least 13 contiguous nucleotides selected from nucleotides 1-1039 of SEQ ID NO:5, at least 651 contiguous nucleotides of SEQ ID NO:7, at least 522 contiguous nucleotides of SEQ ID NO:7, at least 11 contiguous nucleotides selected from nucleotides 1-913 of SEQ ID NO:7, at least 484 contiguous nucleotides of SEQ ID NO:9, at least 317 contiguous nucleotides of SEQ ID NO:9, at least 11 contiguous nucleotides selected from nucleotides 1-216 of SEQ ID NO:9, at least 11 contiguous nucleotides selected from nucleotides 379-812 of SEQ ID NO:9, at least 183 contiguous nucleotides selected from nucleotides 1-984 of SEQ ID NO:9, at least 594 contiguous nucleotides of SEQ ID NO:11, at least 289 contiguous nucleotides of SEQ ID NO: 11, at least 11 contiguous nucleotides selected from nucleotides 1-585 of SEQ ID NO:11, at least 11 contiguous nucleotides selected from nucleotides 853-1120 of SEQ ID NO:11, at least 592 contiguous nucleotides of SEQ ID NO:13, at least 275 contiguous nucleotides of SEQ ID NO:13, at least 11 contiguous nucleotides selected from nucleotides 1-294 of SEQ ID NO:13, at least 537 contiguous nucleotides of SEQ ID NO:15, at least 294 contiguous nucleotides selected from nucleotides 1-1889 of SEQ ID NO:15, at least 171 contiguous nucleotides selected from nucleotides 318-1766 of SEQ ID NO:15, at least 11 contiguous nucleotides selected from nucleotides 1-42 of SEQ ID NO:15, at least 11 contiguous nucleotides selected from nucleotides 478-908 of SEQ ID NO:15, at least 11 contiguous nucleotides selected from nucleotides 1059-1078 of SEQ ID NO:15, at least 205 contiguous nucleotides of SEQ ID NO:17, at least 440 contiguous nucleotides of SEQ ID NO:19, at least 451 contiguous nucleotides of SEQ ID NO:21; at least 11 contiguous nucleotides selected from nucleotides 1-121 of SEQ ID NO:21, at least 11 contiguous nucleotides selected from nucleotides 474-592 of SEQ ID NO:21, at least 351 contiguous nucleotides of SEQ ID NO:23, at least 21 contiguous nucleotides selected from nucleotides 1-1943 of SEQ ID NO:23, at least 11 contiguous nucleotides selected from 1-612 of SEQ ID NO:23, at least 11 contiguous nucleotides selected from nucleotides 611-719 of SEQ ID NO:23, at least 11 contiguous nucleotides selected from nucleotides 713-830 of SEQ ID NO:23, at least 11 contiguous nucleotides selected from nucleotides 830-1933 of SEQ ID NO:23, at least 492 nucleotides of SEQ ID NO:25, at least 11 contiguous nucleotides selected from nucleotides 758-847 of SEQ ID NO:25, at least 1024 contiguous nucleotides of SEQ ID NO:27, at least 347 contiguous nucleotides of SEQ ID NO:29, at least 11 contiguous nucleotides selected from nucleotides 548-601 of SEQ ID NO:29, at least 394 contiguous nucleotides of SEQ ID NO:31, at least 11 contiguous nucleotides selected from nucleotides 1-361 of SEQ ID NO:31, at least 11 contiguous nucleotides selected from nucleotides 1083-1102 of SEQ ID NO:31, at least 492 contiguous nucleotides of SEQ ID NO:33, at least 510 contiguous nucleotides of SEQ ID NO:35, at least 11 contiguous nucleotides selected from nucleotides 1-502 or 505-631 of SEQ ID NO:35, at least 392 contiguous nucleotides of SEQ ID NO:37, at least 11 contiguous nucleotides selected from nucleotides 1-502 of SEQ ID NO:37, at least 11 contiguous nucleotides selected from nucleotides 505-631 of SEQ ID NO:37, at least 559 contiguous nucleotides of SEQ ID NO:39, at least 11 contiguous nucleotides selected from nucleotides 1-92 of SEQ ID NO:39, at least 254 contiguous nucleotides of SEQ ID NO:41, at least 11 contiguous nucleotides selected from nucleotides 1-34 of SEQ ID NO:41, at least 11 contiguous nucleotides selected from nucleotides 55-110 of SEQ ID NO:41, at least 103 contiguous nucleotides of SEQ ID NO:43, at least 11 contiguous nucleotides selected from nucleotides 1-280 of SEQ i) NO:43, at least 11 contiguous nucleotides selected from nucleotides 270-319 of SEQ ID NO:43, at least 11 contiguous nucleotides selected from nucleotides 378-423 of SEQ ID NO:43; at least 11 contiguous nucleotides selected from nucleotides 414-492 of SEQ ID NO:43, at least 11 contiguous nucleotides selected from nucleotides 532-570 of SEQ ID NO:43, at least 11 contiguous nucleotides selected from nucleotides 1086-1152 of SEQ ID NO:43, and the complements thereof.

A further embodiment of the invention is a construct comprising isolated and purified subgenomic polynucleotides of the invention.

Another embodiment of the invention is a host cell comprising a construct of the invention.

Yet another embodiment of the invention is a process for producing a protein. A culture of a host cell comprising a construct of the invention is grown in a suitable culture medium. The protein secreted from the host cell is purified.

Another embodiment of the invention is a polynucleotide array comprising at least one single-stranded polynucleotide which comprises at least 12 contiguous nucleotides of a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, and the complements thereof.

Even another embodiment of the invention is a method of detecting differential gene expression between two biological samples. A first biological sample comprising single-stranded polynucleotide molecules with a first polynucleotide array comprising at least one single-stranded polynucleotide which comprises at least 12 contiguous nucleotides of a nucleotide sequence selected from the group consisting of SEQ ID NOS:1, 3, 5, 7, 9, 11, 13, 15, 17, 19, 21, 23, 25, 27, 29, 31, 33, 35, 37, 39, 41, 43, and the complements thereof. A second biological sample comprising single-stranded polynucleotide molecules is contacted with a second polynucleotide array. The first and second polynucleotide arrays comprise identical single-stranded polynucleotides. A first and second pattern of double-stranded polynucleotides bound to the first and second polynucleotide arrays are detected. A difference between the first and second patterns indicates a gene which is differentially expressed between the first and second biological samples.

Methods are also provided for preventing, treating, or ameliorating a medical condition associated with hematopoiesis or bone marrow morphogenesis, which comprises administering to a mammalian subject a therapeutically effective amount of a composition comprising a protein of the present invention and a pharmaceutically acceptable carrier.

Proteins encoded by polynucleotides of the present invention have potential uses in stimulating blood cell generation in patient receiving cancer chemotherapy, for bone marrow transplantation patient, and for healing fractured bones.

### DETAILED DESCRIPTION OF THE INVENTION

Secreted proteins include proteins which, when expressed in a suitable host cell, are transported across or through a membrane, including transport as a result of signal sequences. Secreted proteins include proteins which are secreted wholly (e.g., soluble proteins) or partially (e.g., receptors) from the cell in which they are expressed. Secreted proteins also include proteins which are transported across the membrane of the endoplasmic reticulum.

Polynucleotides of the invention which encode secreted proteins were isolated from a cDNA library derived from human bone marrow stromal cells. Subgenomic polynucleotides of the invention contain less than a whole chromosome and can be single- or double-stranded. Preferably, the polynucleotides are intron-free. Subgenomic polynucleotides of the invention can comprise all or a portion of a nucleotide sequence disclosed in SEQ ID NOS:1, 3, 5, 7, 9, 11, 13, 15, 17, 19, 21, 23, 25, 27, 29, 31, 33, 35, 37, 39, 41, or 43, as explained in detail below. The complements of these nucleotide sequences are contiguous nucleotide sequences which form Watson-Crick base pairs with a contiguous nucleotide sequence as shown in SEQ ID NOS:1, 3, 5, 7, 9, 11, 13, 15, 17, 19, 21, 23, 25, 27, 29, 31, 33, 35, 37, 39, 41, or 43. These complementary sequences are also subgenomic polynucleotides and can be used, inter alia, to provide antisense oligonucleotides.

Degenerate nucleotide sequences encoding amino acid sequences of proteins of the invention, as well as homologous nucleotide sequences which are at least 65%, 75%, 85%, 90%, 95%, 98%, or 99% identical to the nucleotide sequences shown in NOS:1, 3, 5, 7, 9, 11, 13, 15, 17, 19, 21, 23, 25, 27, 29, 31, 33, 35, 37, 39, 41, and 43, are also subgenomic polynucleotides of the invention.. Percent identity is determined using computer programs which employ the Smith-Waterman homology search algorithm, for example as implemented in the MPSRCH program (Oxford Molecular), using an affine gap search with the following parameters: a gap open penalty of 12 and a gap extension penalty of 1. The Smith-Waterman algorithm is taught in Smith and Waterman, Adv. Appl. Math. (1981) 2:482-489.

Typically, homologous sequences can be confirmed by hybridization under stringent conditions, as is known in the art. For example, using the following wash conditions—2×SSC (0.3 M NaCl 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of subgenomic polynucleotides of the invention can also be identified by making suitable probes or primers and screening cDNA expression libraries from other species, such as mice, monkeys, yeast, or bacteria, as well as human cDNA expression libraries. It is well known that the Tₘ of a double-stranded DNA decreases by 1-1.5° C. with every 1 % decrease in homology (Bonner et al., J. Mol Biol. 81, 123 (1973). Homologous subgenomic polynucleotide species can therefore be identified, for example, by hybridizing a putative homologous polynucleotide with a polynucleotide having a nucleotide sequence disclosed in SEQ ID NOS: 1, 3, 5, 7, 9, 11, 13, 15, 17, 19, 21, 23, 25, 27, 29, 31, 33, 35, 37, 39, 41, or 43 to form a test hybrid, comparing the melting temperature of the test hybrid with the melting temperature of a hybrid comprising a polynucleotide having one of the disclosed nucleotide sequences and a polynucleotide which is perfectly complementary to that sequence, and calculating the number or percent of basepair mismatches within the test hybrid.

Nucleotide sequences which hybridize to the coding sequences shown in SEQ ID NOS:1, 3, 5, 7,9, 11, 13, 15, 17, 19, 21, 23, 25, 27, 29, 31, 33, 35, 37, 39, 41, or 43 or their complements following stringent hybridization and/or wash conditions are also subgenomic polynucleotides of the invention. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a nucleotide sequence shown in SEQ ID NOS:1, 3, 5, 7, 9, 11, 13, 15, 17, 19, 21, 23, 25, 27, 29, 31, 33, 35, 37, 39, 41, or 43 and a polynucleotide sequence which is 65%, 75%, 85%, 90%, 95%, 96%, 97%, 98%, or 99% identical to that sequence can be calculated, for example, using the equation of Bolton and McCarthy, Proc. Natl. Acad Sci. U.S.A. 48, 1390 (1962):
Tₘ=81.5° C.−16.6(log₁₀[Na+])+0.41(% G+C)−0.63(% formamide)−600/l),
where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Subgenomic polynucleotides can be isolated and purified free from other nucleotide sequences using standard nucleic acid purification techniques. For example, restriction enzymes and probes can be used to isolate polynucleotide fragments which comprise nucleotide sequences of the invention. Isolated and purified subgenomic polynucleotides are in preparations which are free or at least 90% free of other molecules.

Complementary DNA (cDNA) molecules with coding sequences corresponding to SEQ ID NOS:1, 3, 5, 7, 9, 11, 13, 15, 17, 19, 21, 23, 25, 27, 29, 31, 33, 35, 37, 39, 41, or 43 are also subgenomic polynucleotides of the invention. cDNA molecules of the invention can be made with standard molecular biology techniques, using human mRNA as a template. cDNA molecules can thereafter be replicated using molecular biology techniques known in the art and disclosed in manuals such as Sambrook et al., 1989. An amplification technique, such as the polymerase chain reaction (PCR), can be used to obtain additional copies of subgenomic polynucleotides of the invention, using either human genomic DNA or cDNA as a template.

Alternatively, synthetic chemistry techniques can be used to synthesize subgenomic polynucleotide molecules of the invention. The degeneracy of the genetic code allows alternate nucleotide sequences to be synthesized which will encode a protein having an amino acid sequence shown in SEQ ID NOS:2, 4, 6, 8, 10, 12, 14, 16, 18, 20, 22, 24, 26, 28, 30, 32, 34, 36, 38, 40, 42, or 44 or a biologically active variant of one of those sequences. All such nucleotide sequences are within the scope of the present invention.

The invention also provides polynucleotide probes which can be used, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides selected from SEQ ID NOS:1, 3, 5, 7, 9, 11, 13, 15, 17, 19, 21, 23, 25, 27, 29, 31, 33, 35, 37, 39, 41, or 43. Polynucleotide probes of the invention can comprise a detectable label, such as a radioisotopic, fluorescent, enzymatic, or chemiluminescent label.

Subgenomic polynucleotides of the invention can be used as primers to obtain additional copies of the polynucleotides. Subgenomic polynucleotides of the invention can also be used to express mRNA, protein, polypeptides, antibodies, or fusion proteins of the invention and to generate antisense oligonucleotides and ribozymes.

Isolated polynucleotides of the invention can be present in constructs, such as DNA or RNA constructs. They can be operably linked to a promoter or other expression control sequence in order to produce proteins of the invention recombinantly. Many suitable expression control sequences, such as the pMT2 or pED expression vectors disclosed in Kaufman et al., Nucleic Acids Res. 19, 4485-4490 (1991), are well known in the art. General methods of expressing recombinant proteins are also well known (see, e.g., Kaufman, METHODS IN ENZYMOLOGY 185, 537-566, 1990). An isolated polynucleotide and a promoter or an expression control sequence are operably linked when the isolated polynucleotide and the promoter or expression control sequence are situated within a construct or cell in such a way that the protein is expressed by a host cell which has been transformed or transfected with the polynucleotide and the promoter or expression control sequence.

For example, a construct of the invention can comprise a promoter which is functional in a particular type of host cell. The skilled artisan can readily select an appropriate promoter from the large number of cell type-specific promoters known and used in the art. The polynucleotide is located downstream from the promoter. Constructs of the invention can also contain a transcription terminator which is functional in the host cell. Transcription of the polynucleotide segment initiates at the promoter. A construct can be linear or circular and can contain sequences, if desired, for autonomous replication.

A variety of host cells are available for use in bacterial, yeast, insect, and human expression systems and can be used to propagate or to express polynucleotides of the invention. Constructs comprising the polynucleotides can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, and calcium phosphate-mediated transfection.

Polynucleotides of the invention can be propagated in constructs and cell lines using techniques well known in the art. Polynucleotides can be on linear or circular molecules. They can be on autonomously replicating molecules or on molecules without replication sequences. They can be regulated by their own or by other regulatory sequences, as are known in the art.

Bacterial systems for expressing polynucleotides of the invention include those described in Chang et al., Nature (1978) 275: 615, Goeddel et al., Nature (1979) 281: 544, Goeddel et al., Nucleic Acids Res. (1980) 8: 4057, EP 36,776, U.S. 4,551,433, deBoer et al., Proc. Natl. Acad. Sci. USA (1983) 80: 21-25, and Siebenlist et al., Cell (1980) 20: 269.

Expression systems in yeast include those described in Hinnen et al., Proc. Natl. Acad. Sci. USA (1978) 75: 1929; Ito et al., J. Bacteriol. (1983) 153: 163; Kurtz et al., Mol. Cell. Biol. (1986) 6: 142; Kunze et al., J. Basic Microbiol. (1985) 25: 141; Gleeson et al., J. Gen. Microbiol. (1986) 132: 3459, Roggenkamp et al., Mol. Gen. Genet. (1986) 202: 302) Das et al., J. Bacteriol. (1984) 158: 1165; De Louvencourt et al., J. Bacteriol. (1983) 154: 737, Van den Berg et al., Bio/Technology (1990) 8: 135; Kunze et al., J. Basic Microbiol. (1985) 25: 141; Cregg et al., Mol. Cell. Biol. (1985) 5: 3376, U.S. Pat. No. 4,837,148, U.S. Pat. No. 4,929,555; Beach and Nurse, Nature (1981) 300: 7 06; Davidow et al, Curr. Genet. (1985) 10:3 80, Gaillardin et al., Curr. Genet: (1985) 10: 49, Ballance et al., Biochem. Biophys. Res. Commun. (1983) 112: 284-289; Tilburn et al., Gene (1983) 26: 205-221, Yelton et al., Proc. Natl. Acad. Sci. USA (1984) 81: 1470-1474, Kelly and Hynes, EMBO J. (1985) 4: 475479; EP 244,234; and WO 91/00357.

Expression of polynucleotides of the invention in insects can be carried out as described in U.S. Pat. No. 4,745,051, Friesen et al. (1986) “The Regulation of Baculovirus Gene Expression” in: THE MOLECULAR BIOLOGY OF BACULOVIRUSES (W. Doerfler, ed.), EP 127,839, EP 155,476, and Vlak et al., J Gen. Virol. (1988) 69: 765-776, Miller et al., Ann. Rev. Microbiol. (1988) 42: 177, Carbonell et al., Gene (1988) 73: 409, Maeda et al., Nature (1985) 315: 592-594, Lebacq-Verheyden et al., Mol. Cell. Biol. (1988) 8: 3129; Smith et al., Proc. Natl. Acad. Sci. USA (1985) 82: 8404, Miyajima et al., Gene (1987) 58: 273; and Martin et al., DNA (1988) 7: 99. Numerous baculoviral strains and variants and corresponding permissive insect host cells from hosts are described in Luckow et al., Bio/Technology (1988) 6: 47-55, Miller et al., in GENETIC ENGINEERING (Setlow, J. K. et al. eds.), Vol. 8 (Plenum Publishing, 1986), pp. 277-279, and Maeda et al., Nature, (1985) 315: 592-594.

Mammalian expression of polynucleotides can be achieved as described in Dijkema et al., EMBO J (1985) 4: 761, Gorman et al., Proc. Natl. Acad. Sci. USA (1982b) 79: 6777, Boshart et al., Cell (1985) 41: 521 and U.S. Pat. No. 4,399,216. Other features of mammalian expression can be facilitated as described in Ham and Wallace, Meth. Enz. (1979) 58: 44, Barnes and Sato, Anal. Biochem. (1980) 102: 255, U.S. Pat. No. 4,767,704, U.S. Pat. No. 4,657,866, U.S. Pat. No. 4,927,762, U.S. Pat. No. 4,560,655, WO 90/103430, WO 87/00195, and U.S. RE 30,985.

Polynucleotides of the invention can also be used in gene delivery vehicles, for the purpose of delivering an mRNA or oligonucleotide (either with the sequence of a native mRNA or its complement), fill-length protein, fusion protein, polypeptide, or ribozyme, or single-chain antibody, into a cell, preferably a eukaryotic cell. According to the present invention, a gene delivery vehicle can be, for example, naked plasmid DNA, a viral expression vector comprising a polynucleotide of the invention, or a polynucleotide of the invention in conjunction with a liposome or a condensing agent.

In one embodiment of the invention, the gene delivery vehicle comprises a promoter and one of the polynucleotides disclosed herein. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter.

A gene delivery vehicle can comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and W091/02805).

Particularly preferred retroviruses are derived from retroviruses which include avian leukosis virus (ATCC Nos. VR-535 and VR-247), bovine leukemia virus (VR-1315), murine leukemia virus (MLV), mink-cell focus-inducing virus (Koch et al., J Vir. 49:828, 1984; and Oliff et al., J. Vir. 48:542, 1983), murine sarcoma virus (ATCC Nos. VR-844, 45010 and 45016), reticuloendotheliosis virus (ATCC Nos VR-994, VR-770 10 and 45011), Rous sarcoma virus; Mason-Pfizer monkey virus, baboon endogenous virus, endogenous feline retrovirus (e.g., RD114), and mouse or rat gL30 sequences used as a retroviral vector. Particularly preferred strains of MLV from which recombinant retroviruses can be generated include 4070A and 1504A (Hartley and Rowe, J. Vir. 19:19; 1976), Abelson (ATCC No. VR-999), Friend (ATCC No. VR-245), Graffi (Ru et al., J Vir. 67:4722, 1993; and Yantchev Neoplasma 26:397, 1979), Gross (ATCC No. VR-590), Kirsten (Albino et al., J. Exp. Med. 164:1710, 1986), Harvey sarcoma virus (Manly et al., J. Vir. 62:3540, 1988; and Albino et al., J. Exp. Med 164:1710, 1986) and Rauscher (ATCC No. VR-998), and Moloney MLV (ATCC No. VR-190). A particularly preferred non-mouse retrovirus is Rous sarcoma virus. Preferred Rous sarcoma viruses include Bratislava (Manly et al., J. Vir. 62:3540, 1988; and Albino et al., J. Exp. Med. 164:1710, 1986), Bryan high titer (e.g., ATCC Nos. VR-334, VR-657, VR-726, VR-659, and VR-728), Bryan standard (ATCC No. VR-140), Carr-Zilber (Adgighitov et al., Neoplasma 27:159, 1 980), Engelbreth-Holm (Laurent et al., Biochem Biophys Acta 908:241, 1987), Harris, Prague (e.g., ATCC Nos. VR-772, and 45033), and Schmidt-Ruppin (e.g. ATCC Nos. VR-724, VR-725, VR-354) viruses.

Any of the above retroviruses can be readily utilized in order to assemble or construct retroviral gene delivery vehicles given the disclosure provided herein and standard recombinant techniques (e.g., Sambrook et al., 1989, and Kunkle, Proc. Natl. Acad. Sci. U.S.A. 82:488, 1985) known in the art. Portions of retroviral expression vectors can be derived from different retroviruses. For example, retrovector LTRs can be derived from a murine sarcoma virus, a tRNA binding site from a Rous sarcoma virus, a packaging signal from a murine leukemia virus, and an origin of second strand synthesis from an avian leukosis virus. These recombinant retroviral vectors can be used to generate transduction competent retroviral vector particles by introducing them into appropriate packaging cell lines (see Ser. No. 07/800,921, filed Nov. 29, 1991): Recombinant retroviruses can be produced which direct the site-specific integration of the recombinant retroviral genome into specific regions of the host cell DNA. Such site-specific integration can be mediated by a chimeric integrase incorporated into the retroviral particle (see Ser. No. 08/445,466 filed May 22, 1995). It is preferable that the recombinant viral gene delivery vehicle is a replication-defective recombinant virus.

Packaging cell lines suitable for use with the above-described retroviral gene delivery vehicles can be readily prepared (see Ser. No. 08/240,030, filed May 9, 1994; see also WO 92/05266) and used to create producer cell lines (also termed vector cell lines or “VCLs”) for production of recombinant viral particles. In particularly preferred embodiments of the present invention, packaging cell lines are made from human (e.g., HT1080 cells) or mink parent cell lines, thereby allowing production of recombinant retroviral gene delivery vehicles which are capable of surviving inactivation in human serum. The construction of recombinant retroviral gene delivery vehicles is described in detail in WO 91/02805. These recombinant retroviral gene delivery vehicles can be used to generate transduction competent retroviral particles by introducing them into appropriate packaging cell lines (see Ser. No. 07/800,921). Similarly, adenovirus gene delivery vehicles can also be readily prepared and utilized given the disclosure provided herein (see also Berkner, Biotechniques 6:616-627,1988, and Rosenfeld et al., Science 252:431-434, 1991, WO 93/07283, WO 93/06223, and WO 93/07282).

A gene delivery vehicle can also be a recombinant adenoviral gene delivery vehicle. Such vehicles can be readily prepared and utilized given the disclosure provided herein (see Berkner, Biotechniques 6:616, 1988, and Rosenfeld et al., Science 252:431, 1991, WO 93/07283, WO 93/06223, and WO 93/07282). Adeno-associated viral gene delivery vehicles can also be constructed and used to deliver proteins or polynucleotides of the invention to cells in vitro or in vivo. The use of adeno-associated viral gene delivery vehicles in vitro is described in Chatterjee et al., Science 258: 1485-1488 (1992), Walsh et al., Proc. Nat'l. Acad. Sci. 89: 7257-7261 (1992), Walsh et al., J. Clin. Invest. 94: 1440-1448 (1994), Flotte et al., J. Biol Chem. 268: 3781-3790 (1993), Ponnazhagan et al., J. Exp. Med. 179: 733-738 (1994), Miller et al., Proc. Nat'l Acad. Sci. 91: 10183-10187 (1994), Einerhand et al., Gene Ther. 2: 336-343 (1995), Luo et al., Exp. Hematol 23: 1261-1267 (1995), and Zhou et al., Gene Therapy 3: 223-229 (1996). In vivo use of these vehicles is described in Flotte et al., Proc. Nat'l Acad Sci. 90: 10613-10617 (1993), and Kaplitt et al., Nature Genet. 8:148-153 (1994).

In another embodiment of the invention, a gene delivery vehicle is derived from a togavirus. Preferred togaviruses include alphaviruses; in particular those described in U.S. Ser. No. 08/405,627, filed Mar. 15, 1995, WO 95/07994. Alpha viruses, including Sindbis and ELVS viruses can be gene delivery vehicles for polynucleotides of the invention. Alpha viruses are described in WO 94/21792, WO 92/10578 and WO 95/07994. Several different alphavirus gene delivery vehicle systems can be constructed and used to deliver polynucleotides to a cell according to the present invention.

Representative examples of such systems include those described in U.S. Pat. Nos. 5,091,309 and 5,217,879. Particularly preferred alphavirus gene delivery vehicles for use in the present invention include those which are described in WO 95/07994, and U.S. Ser. No. 08/405,627.

Preferably, the recombinant viral vehicle is a recombinant alphavirus viral vehicle based on a Sindbis virus. Sindbis constructs, as well as numerous similar constructs, can be readily prepared essentially as described in U.S. Ser. No. 08/198,450. Sindbis viral gene delivery vehicles typically comprise a 5′ sequence capable of initiating Sindbis virus transcription, a nucleotide sequence encoding Sindbis non-structural proteins, a viral junction region inactivated so as to prevent fragment transcription, and a Sindbis RNA polyrnerase recognition sequence. Optionally, the viral junction region can be modified so that polynucleotide transcription is reduced, increased, or maintained. As will be appreciated by those in the art, corresponding regions from other alphaviruses can be used in place of those described above.

The viral junction region of an alphavirus-derived gene delivery vehicle can comprise a first viral junction region which has been inactivated in order to prevent transcription of the polynucleotide and a second viral junction region which has been modified such that polynucleotide transcription is reduced. An alphavirus-derived vehicle can also include a 5′ promoter capable of initiating synthesis of viral RNA from cDNA and a 3′ sequence which controls transcription termination.

Other recombinant togaviral gene delivery vehicles which can be utilized in the present invention include those derived from Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309 and 5,217,879 and in WO 92/10578. The Sindbis vehicles described above, as well as numerous similar constructs, can be readily prepared essentially as described in U.S. Ser. No. 08/198,450.

Other viral gene delivery vehicles suitable for use in the present invention include, for example, those derived from poliovirus (Evans et al., Nature 339:385, 1989, and Sabin et al., J. Biol Standardization 1:115, 1973) (ATCC VR-58); rhinovirus (Arnold et al., J. Cell. Biochem. L401, 1990) (ATCC VR-1110); pox viruses, such as canary pox virus or vaccinia virus (Fisher-Hoch et al., PROC. NATL. ACAD. SC. U.S.A. 86:317, 1989; Flexner et al., Ann. N.Y. Acad. Sci. 569:86, 1989; Flexner et al., Vaccine 8:17, 1990; U.S. Pat. No. 4,603,112 and U.S. Pat. No. 4,769,330; WO 89/01973) (ATCC VR111; ATCC VR-2010); SV40 (Mulligan et al., Nature 277:108, 1979) (ATCC VR-305), (Madzak et al., J. Gen. Vir. 73:1533, 1992); influenza virus (Luytjes et al., Cell 59:1107, 1989; McMicheal et al., The New England Journal of Medicine 309:13, 1983; and Yap et al., Nature 273:238, 1978) (ATCC VR-797); parvovirus such as adenoassociated virus (Samulski et al., J. Vir. 63:3822, 1989, and Mendelson et al., Virology 166:154, 1988) (ATCC VR-645); herpes simplex virus (Kit et al., Adv. Exp. Med. Biol. 215:219, 1989) (ATCC VR-977; ATCC VR-260); Nature 277:108, 1979); human immunodeficiency virus (EPO 386,882, Buchschacher et al., J. Vir. 66:2731, 1992); measles virus (EPO 440,219) (ATCC VR-24); A (ATCC VR-67; ATCC VR-1247), Aura (ATCC VR-368), Bebaru virus (ATCC VR-600; ATCC VR-1240), Cabassou (ATCC VR-922), Chikungunya virus (ATCC VR-64; ATCC VR-1241), Fort Morgan (ATCC VR-924), Getah virus (ATCC VR-369; ATCC VR-1243), Kyzylagach (ATCC VR-927), Mayaro (ATCC VR-66), Mucambo virus (ATCC VR-580;

ATCC VR-1244), Ndumu (ATCC VR-371), Pixuna virus (ATCC VR-372; ATCC VR-1245), Tonate (ATCC VR-925), Triniti (ATCC VR-469), Una (ATCC VR-374), Whataroa (ATCC VR-926), Y-62-33 (ATCC VR-375), ONyong virus, Eastern encephalitis virus (ATCC VR-65; ATCC VR-1242), Western encephalitis virus (ATCC VR-70; ATCC VR-1251; ATCC VR-622; ATCC VR-1252), and coronavirus Hamre et al., Proc. Soc. Exp. Biol. Med. 121:190,1966) (ATCC VR-740).

A polynucleotide of the invention can also be combined with a condensing agent to form a gene delivery vehicle. In a preferred embodiment, the condensing agent is a polycation, such as polylysine, polyarginine, polyornithine, protamine, spermine, spermidine, and putrescine. Many suitable methods for making such linkages are known in the art (see, for example, Ser. No.08/366,787, filed Dec. 30, 1994).

In an alternative embodiment, a polynucleotide is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier which sequesters and protects its contents, for example, from degradative enzymes. Additionally, because a liposome is a synthetic structure, specially designed liposomes can be produced which incorporate desirable features. See Stryer, Biochemistry, pp. 236-240, 1975 (W.H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PROC. NATL. ACAD. S CI. U.S.A. 84:7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising polynucleotides such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et a., Proc. Natl. Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl)-N,N,N-triethylammonium (DOTMA) liposomes are available under the trademark Lipofectin, from GIBCO BRL, Grand Island, N.Y. See also Felgner et al., Proc. Natl. Acad. Sci. USA 91:5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylarnmonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., METHODS OF IMMUNOLOGY (1983), Vol. 101, pp. 512-527; Szoka et al., Proc. Natl. Acad. Sci. USA 87:3410-3414, 1990; Papahadjopoulos et al., Biochim. Biophys. Acta 394:483, 1975; Wilson et al., Cell 17:77, 1979; Deamer and Bangham, Biochim. Biophys. Acta 443:629, 1976; Ostro et al., Biochem. Biophys. Res. Commun. 76:836, 1977; Fraley et al., Proc. Nat. Acad. Sci. USA 76:3348, 1979; Enoch and Strittmatter, Proc. Natl. Acad. Sci. USA 76:145,1979; Fraley et al., J. Biol. Chem. 255:10431, 1980; Szoka and Papahadjopoulos, Proc. Natl. Acad. Sci. USA 75:145, 1979; and Schaefer-Ridder et al., Science 215:166, 1982.

In addition, lipoproteins can be included with a polynucleotide of the invention for delivery to a cell. Examples of such lipoproteins include chylomicrons, HDL, IDL, LDL, and VLDL. Mutants, fragments, or fusions of these proteins can also be used. Modifications of naturally occurring lipoproteins can also be used, such as acetylated LDL. These lipoproteins can target the delivery of polynucleotides to cells expressing lipoprotein receptors. Preferably, if lipoproteins are included with a polynucleotide, no other targeting ligand is included in the composition.

In another embodiment, naked polynucleotide molecules are used as gene delivery vehicles, as described in WO 90/11092 and U.S. Pat. No. 5,580,859. Such gene delivery vehicles can be either DNA or RNA and, in certain embodiments, are linked to killed adenovirus. Curiel et al., Hum. Gene. Ther. 3:147-154, 1992. Other suitable vehicles include DNA-ligand (Wu et al., J. Biol. Chem. 2 64:16985-16987, 1989), lipid-DNA combinations (Felgner et al., Proc. Nat. Acad. Sci. USA 84:7413-7417, 1989), liposomes (Wang et al., Proc. Nat. Acad. Sci. 84:7851-7855, 1987) and microprojectiles (Williams et al., Proc. Natl. Acad. Sci. 88:2726-2730, 1991).

One can increase the efficiency of naked polynucleotide uptake into cells by coating the polynucleotides onto biodegradable latex beads. This approach takes advantage of the observation that latex beads, when incubated with cells in culture, are efficiently transported and concentrated in the perinuclear region of the cells. The beads will then be transported into cells when injected into muscle. Polynucleotide-coated latex beads will be efficiently transported into cells after endocytosis is initiated by the latex beads and thus increase gene transfer and expression efficiency. This method can be improved further by treating the beads to increase their hydrophobicity, thereby facilitating the disruption of the endosome and release of polynucleotides into the cytoplasm.

One polynucleotide of the invention is designated hCornichon. The nucleotide sequence of hCornichon is shown in SEQ ID NO:1. hCornichon cDNA represents a transcript of 1325 nucleotides with a translation stop codon (TAG) at position 428, a polyadenylation signal (AATAAA) (SEQ ID NO:45) at position 1292, and a poly(A) tail at position 1316. The DNA sequence between nucleotides 2 and 427 encodes a protein of 142 amino acids, as shown in SEQ ID NO:2. A potential signal peptide is located in the first 28 amino acid residues. An hCornichon polynucleotide can comprise at least 499, 550, 600, 700, 750, 800, 850, 850, 900, 950, 1000, 1100, 1141, 1150, 1200, or 1250 nucleotides of SEQ ID NO:1 or the complements thereof.

Another polynucleotide of the invention is designated BMS46. The nucleotide sequence of BMS46 is shown in SEQ ID NO:3. BMS46 cDNA represents a transcript of 1277 nucleotides with a translation start codon (ATG) at position 656, a translation stop codon (TAG) at position 1223, a polyadenylation signal (AATAAA) (SEQ ID NO:45) at position 1243, and a poly(A) tail at position 1260. The DNA sequence between nucleotides 656 and 1222 encodes a protein of 189 amino acid residues, as shown in SEQ ID NO:4. A potential signal peptide is located in the first 47 amino acid residues. A BMS46 polynucleotide can comprise at least 474, 475, 476, 477, 500, 550, 600, 650, 700, 750, 800, 850, 900, 950, 1000, 1050, 1150, 1200, or 1250 contiguous nucleotides of SEQ ID NO:3, or at least 313, 314, 315, or 316 contiguous nucleotides selected from nucleotides 1-1001 of SEQ ID NO:3, or the complements thereof.

The nucleotide sequence of another polynucleotide of the invention, termed BMS112, is shown in SEQ ID NO:5. BMS112 cDNA represents a transcript of 1610 nucleotides with a translation start codon (ATG) at position 132, a translation stop codon (TGA) at position 1251, a polyadenylation signal (AATAAA) (SEQ ID NO:45) at position 1516, and a poly(A) tail at position 1594. The DNA sequence between nucleotides 132 and 1250 encodes a polypeptide of 373 amino acid residues (SEQ ID NO:6). A BMS112 polynucleotide can comprise at least 538, 600, 700, 751, 800, 850, 900, 950, 1000, 1200, 1300, 1400, 1500 or 1600 contiguous nucleotides of SEQ ID NO:5, at least 11, 12, 13, 14, 15, 20, 25, 30, 35, 40, or 50 contiguous nucleotides selected from nucleotides 1-946, at least 13 contiguous nucleotides selected from nucleotides 1-1039 of SEQ ID NO:5, or the complements thereof.

Yet another polynucleotide of the invention has the nucleotide sequence shown in SEQ ID NO:7 and is designated BMS118. BMS118 cDNA represents a transcript of 1499 nucleotides with a translation start codon (ATG) at position 140, a translation stop codon (TAA) at position 1358, a polyadenylation signal (AATAAA) (SEQ ID NO:45) at position 1463, and a poly(A) tail at position 1482. The DNA sequence between nucleotides 140 and 1357 encodes a polypeptide of 406 amino acid residues (SEQ ID NO:8). The potential signal peptide of the BMS118 protein is located in the first 29 amino acids. A BMS118 polynucleotide can comprise at least 522, 550, 600, 651, 700, 750, 800, 850, 900, 950, 1000, 1050, 1100, 1150, 1200, 1250, 1300, 1350, 1400, or 1450 contiguous nucleotides of SEQ ID NO:7, at least 11, 12, 13, 14, 15, 20, 25, 30, 35, 40, or 50 contiguous nucleotides selected from nucleotides 1-913 of SEQ ID NO:7, or the complements thereof.

Another polynucleotide of the invention has the nucleotide sequence shown in SEQ ID NO:9 and is designated BMS164. BMS164 cDNA represents a transcript of 1272 nucleotides with a translation start codon (ATG) at position 313 and a translation stop codon (TAG) at position 1186. The DNA sequence between nucleotides 313 and 1185 encodes a polypeptide of 291 amino acid residues (SEQ ID NO:10). A BMS 164 polynucleotide can comprise at least 317, 400, 484, 500, 600, 700, 800, 900, 1000, 1100, or 1200 contiguous nucleotides of SEQ ID NO:9, at least 183 contiguous nucleotides selected from nucleotides 1-984 of SEQ ID NO:9, or at least 11, 12, 13, 14, 15, 20, 25, 30, 35, 40, or 50 contiguous nucleotides selected from nucleotides 1-216 or 379-812 of SEQ ID NO:9, or the complements thereof.

Another polynucleotide of the invention, BMS192, has the nucleotide sequence shown in SEQ ID NO:11. BMS192 cDNA represents a transcript of 1585 nucleotides with a translation start codon (ATG) at position 41, a translation stop codon (TGA) at position 1190, a polyadenylation signal (AATAAA) (SEQ ID NO:45) at position 1439, and a poly(A) tail at position 1574. The DNA sequence between nucleotides 41 and 1189 encodes a polypeptide of 383 amino acid residues (SEQ ID NO:12). The potential signal peptide of the BMS192 protein is located in the first 19 amino acids. A BMS192 polynucleotide can comprise at least 289, 300, 400, 500, 594, 600, 700, 800, 900, 1000, 1100, 1200, 1300, 1400, or 1500 contiguous nucleotides of SEQ ID NO:11, at least 11, 12, 13, 14, 15, 20, 25, 30, 35, 40, or 50 contiguous nucleotides selected from nucleotides 1-585 or 853-1120 of SEQ ID NO:11, or the complements thereof.

Another polynucleotide of the invention, BMS227, has the nucleotide sequence shown in SEQ ID NO:13. BMS227 cDNA represents a transcript of 1071 nucleotides with a translation start codon (ATG) at position 151, a translation stop codon (TGA) at position 934, a polyadenylation signal (AATAAA) (SEQ ID NO:45) at position 1018, and a poly(A) tail at position 1053. The DNA sequence between nucleotides 151 and 933 encodes a polypeptide of 261 amino acid residues (SEQ ID NO:14). The potential signal peptide of the BMS227 protein is located in the first 32 amino acids. A BMS227 polynucleotide can comprise 275, 300, 400, 500, 592, 600, 700, 800, 900, or 1000 contiguous nucleotides of SEQ ID NO:13, at least 11, 12, 13, 14, 15, 20, 25, 30, 35, 40, or 50 contiguous nucleotides selected from nucleotides 1-294 of SEQ ID NO:13, or the complements thereof.

Yet another polynucleotide of the invention is designated BMS115. The nucleotide sequence of BMS 115 is shown in SEQ ID NO: 15. BMS 115 cDNA represents a transcript of 2520 nucleotides with a translation start codon (ATG) at position 1, a translation stop codon at position 1666, a polyadenylation signal (AATAAA) (SEQ ID NO:45) at position 2470, and a poly(A) tail at position 2503. The DNA sequence between nucleotides 1 and 1665 encodes a protein of 555 amino acids, as shown in SEQ ID NO:16. A potential signal peptide is located in the first 31 amino acid residues. A BMS115 polynucleotide can comprise at least 537, 600, 700, 800, 900, 1000, 1250, 1500, 1750, 2000, 2250, or 2500 contiguous nucleotides of SEQ ID NO:15, at least 294 contiguous nucleotides selected from nucleotides 1-1889 of SEQ ID NO:15, at least 171 contiguous nucleotides selected from nucleotides 318-1766 of SEQ ID NO:15, or at least 11, 12, 13, 14, 15, 20, 25, 30, 35, 40, or 50 contiguous nucleotides selected from nucleotides 1-42, 478-908, or 1059-1078 of SEQ ID NO:15, or the complements thereof.

Yet another polynucleotide of the invention is designated BMS143. The nucleotide sequence of BMS143 is shown in SEQ ID NO:17. BMS143 cDNA represents a transcript of 1245 nucleotides with a translation start codon (ATG) at position 89, a translation stop codon at position 785, a polyadenylation signal (AATAAA) (SEQ ID NO:45) at position 1199, and a poly(A) tail at position 1231. The DNA sequence between nucleotides 89 and 784 encodes a protein of 232 amino acids, as shown in SEQ ID NO:18. A potential signal peptide is located in the first 54 amino acid residues. A BMS143 polynucleotide can comprise at least 205, 300, 400, 500, 600, 700, 800, 900, 1000, 1100, or 1200 contiguous nucleotides of SEQ ID NO:17, or the complements thereof.

Yet another polynucleotide of the invention is designated BMS155. The nucleotide sequence of BMS155 is shown in SEQ ID NO:19. BMS155 cDNA represents a transcript of 1030 nucleotides with a translation start codon (ATG) at position 4, a translation stop codon at position 451, a polyadenylation signal (AATAAA) (SEQ ID NO:45) at position 987, and a poly(A) tail at position 1016. The DNA sequence between nucleotides 4 and 450 encodes a protein of 149 amino acids, as shown in SEQ ID NO:20. A potential signal peptide is located in the first 47 amino acid residues. A BMS155 polynucleotide can comprise at least 440, 500, 600, 700, 800, 900, or 1000 contiguous nucleotides of SEQ ID NO:19 or the complements thereof.

Yet another polynucleotide of the invention is designated BMS208. The nucleotide sequence of BMS208 is shown in SEQ ID NO:21. BMS208 cDNA represents a transcript of 1563 nucleotides with a translation start codon (ATG) at position 255, a translation stop codon at position 756, a polyadenylation signal (AATAAA) (SEQ ID NO:45) at position 1531, and a poly(A) tail at position 1550. The DNA sequence between nucleotides 255 and 755 encodes a protein of 167 amino acids, as shown in SEQ ID NO:22. A potential signal peptide is located in the first 62 amino acid residues. A BMS208 polynucleotide can comprise at least 451, 500, 600, 750, 1000, 1250, or 1500 contiguous nucleotides of SEQ ID NO:21, at least 11, 12, 13, 14, 15, 20, 25, 30, 35, 40, or 50 contiguous nucleotides selected from nucleotides 1-121 or 474-592 of SEQ ID NO:21, or the complements thereof.

Yet another polynucleotide of the invention is designated BMS235. The nucleotide sequence of BMS235 is shown in SEQ ID NO:23. BMS235 cDNA represents a transcript of 2590 nucleotides with a translation start codon (ATG) at position 29, a translation stop codon at position 872, and a poly(A) tail at position 1526. The DNA sequence between nucleotides 29 and 871 encodes a protein of 281 amino acids, as shown in SEQ ID NO:24. A potential signal peptide is located in the first 25 amino acid residues. A BMS235 polynucleotide can comprise at least 351 contiguous nucleotides of SEQ ID NO:23, at least 11, 12, 13, 14, 15, 20, 25, 30, 35, 40, or 50 contiguous nucleotides selected from nucleotides 1-612, 611-719, 713-830, or 830-1933 of SEQ ID NO:23, at least 21 contiguous nucleotides selected from nucleotides 1-1943 of SEQ ID NO:23, or the complements thereof.

Yet another polynucleotide of the invention is designated BMS240. The nucleotide sequence of BMS240 is shown in SEQ ID NO:25. BMS240 cDNA represents, a transcript of 1668 nucleotides with a translation start codon (ATG) at position 99, a translation stop codon at position 807, a polyadenylation signal (AATAAA) (SEQ ID NO:45) at position 1626, and a poly(A) tail at position 1655. The DNA sequence between nucleotides 99 and 806 encodes a protein of 236 amino acids, as shown in SEQ ID NO:26. A BMS240 polynucleotide can comprise at least 492, 500, 600, 750, 1000, 1250, 1500, or 1600 contiguous nucleotides of SEQ ID NO:25, at least 11, 12, ,13, 14, 15, 20, 25, 30, 35, 40, or 50 contiguous nucleotides selected from nucleotides 758-847 of SEQ ID NO:25, or the complements thereof.

Yet another polynucleotide of the invention is designated BMS53. The nucleotide sequence of BMS53 is shown in SEQ ID NO:27. BMS53 cDNA represents a transcript of 1697 nucleotides with a translation start codon (ATG) at position 29, a translation stop codon at position 1427, a polyadenylation signal (ATTAAA) (SEQ ID NO:46) at position 1659, and a poly(A) tail at position 1682. The DNA sequence between nucleotides 29 and 1426 encodes a polypeptide of 466 amino acid residues, as shown in SEQ ID NO:28. A BMS53 polynucleotide can comprise at least 1024, 1100, 1200, 1300, 1400, 1500, or 1600 contiguous nucleotide of SEQ ID NO:27 or the complements thereof.

Yet another polynucleotide of the invention is designated BMS100. The nucleotide sequence of BMS100 is shown in SEQ ID NO:29. BMS100 cDNA represents a transcript of 1830 nucleotides with a translation start codon (ATG) at position 218, a translation stop codon at position 851, a polyadenylation signal (AATAAA) (SEQ ID NO:35) at position 1792, and a poly(A) tail at position 1811. The DNA sequence between nucleotides 218 and 850 encodes a protein of 211 amino acids, as shown in SEQ ID NO:30. A potential signal peptide is located in the first 18 amino acid residues. A BMS100 polynucleotide can comprise at least 347, 400, 500, 600, 700, 800, 900, 1000, 1100, 1200, 1300, 1400, 1500, 1600, 1700, or 1800 contiguous nucleotides of SEQ ID NO:29, at least 11, 12, 13, 14, 15, 20, 25, 30, 35, 40, or 50 contiguous nucleotides selected from nucleotides 548-601 of SEQ ID NO:29, or the complements thereof.

Yet another polynucleotide of the invention is designated BMS199. The nucleotide sequence of BMS199 is shown in SEQ ID NO:31. BMS199 cDNA represents a transcript of 1102 nucleotides with a translation start codon (ATG) at position 267, a translation stop codon at position 990, a polyadenylation signal (AATAAA) (SEQ ID NO:45) at position 1072, and a poly(A) tail at position 1089. The DNA sequence between nucleotides 267 and 989 encodes a protein of 241 amino acids, as shown in SEQ ID NO:32. A potential signal peptide is located in the first 32 amino acid residues. A BMS199 polynucleotide can comprise at least 394, 400, 500, 600, 700, 800, 900, 1000, or 1100 contiguous nucleotides of SEQ ID NO:31, at least 11, 12, 13, 14, 15, 20, 25, 30, 35, 40, or 50 contiguous nucleotides selected from nucleotides 1-361 or 1083-1102 of SEQ ID NO:3 1, or the complements thereof.

Yet another polynucleotide of the invention is designated BMS206. The nucleotide sequence of BMS206 is shown in SEQ ID NO:33. BMS206 cDNA represents a transcript of 966 nucleotides with a translation start codon (ATG) at position 36, a translation stop codon at position 585, a polyadenylation signal (AATAAA) (SEQ ID NO:45) at position 920, and a poly(A) tail at position 949. The DNA sequence between nucleotides 36 and 584 encodes a protein of 183 amino acids, as shown in SEQ ID NO:34. A BMS206 polynucleotide can comprise at least 492, 500, 600, 700, 800, or 900 contiguous nucleotides of SEQ ID NO:33 or the complements thereof.

Yet another polynucleotide of the invention is designated BMS242. The nucleotide sequence of BMS242 is shown in SEQ ID NO:35. BMS242 cDNA represents a transcript of 1570 nucleotides with a translation start codon (ATG) at position 76, a translation stop codon at position 1030, and a poly (1) tail at position 1562. The DNA sequence between nucleotides 76 and 1029 encodes a protein of 318 amino acid residues, as shown in SEQ ID NO:36. A BMS242 polynucleotide can comprise at least 510, 600, 700, 800, 900, 1000, 1100, 1200, 1300, 1400, or 1500 contiguous nucleotides of SEQ ID NO:35, at least 11, 12, 13, 14, 15, 20, 25, 30, 35, 40, or 50, contiguous nucleotides selected from nucleotides 1-502 or 505-631 of SEQ ID NO:35, or the complements thereof.

Yet another polynucleotide of the invention is termed BMS37. The nucleotide sequence of BMS37 is shown in SEQ ID NO:37. BMS37 cDNA represents a transcript of 1542 nucleotides with a translation start codon (ATG) at position 121, a translation stop codon at position 1105, a polyadenylation signal (AATAAA) (SEQ ID NO:45) at position 1508, and a poly(A) tail at position 1526. The DNA sequence between nucleotides 121 and 1104 encodes a protein of 328 amino acid residues, as shown in SEQ ID NO:38. The potential signal peptide the BMS37 protein is located in the first 20 amino acids. A BMS37 polynucleotide can comprise at least 392, 400, 500, 600, 700, 800, 900, 1000, 1100, 1200, 1300, 1400, or 1500 contiguous nucleotides of SEQ ID NO:37, at least 11, 12, 13, 14, 15, 20, 25, 30, 35, 40, or 50 contiguous nucleotides selected from nucleotides 1-502 or 505-631 of SEQ ID NO:37, or the complements thereof.

Yet another polynucleotide of the invention is designated BMS42. The nucleotide sequence of BMS42 is shown in SEQ ID NO:39. BMS42 cDNA represents a transcript of 1990 nucleotides with a translation start codon (ATG) at position 104, a translation stop codon at position 1615, a polyadenylation signal (AATAAA) (SEQ ID NO:45) at position 1952, and a poly(A) tail at position 1971. The DNA sequence between nucleotides 104 and 1614 encodes a protein of 504 amino acids, as shown in SEQ ID NO:40. A potential signal peptide is located in the first 67 amino acids. A BMS42 polynucleotides can comprise at least 559, 600, 700, 800, 900, 10000, 1250, 1500, 1750, 1800, or 1900 contiguous nucleotides of SEQ ID NO:39, at least 11, 12, 13, 14, 15, 20, 25, 30, 35, 40, or 50 contiguous nucleotides selected from nucleotides 1-92 of SEQ ID NO:39, or the complements thereof.

Yet another polynucleotide of the invention is designated BMS60. The nucleotide sequence of BMS60 is shown in SEQ ID NO:41. BMS60 cDNA represents a transcript of 684 nucleotides with a translation start codon (ATG) at position 7, a translation stop codon at position 445, a polyadenylation signal (AATAAA) (SEQ ID NO:45) at position 644, and a poly(A) tail at position 667. The DNA sequence between nucleotides 7 and 444 encodes a protein of 146 amino acid residues, as shown in SEQ ID NO:42. A potential signal peptide is located in the first 20 amino acids. A BMS60 polynucleotide can comprise at least 254, 300, 350, 400, 450, 500, 550, 600, or 650 contiguous nucleotides of SEQ ID NO:41, at least 11, 12, 13, 14, 15, 20, 25, 30, 35, 40, or 50 contiguous nucleotides selected from nucleotides 1-34 or 55-110 of SEQ ID NO:41, or the complements thereof.

Yet another polynucleotide of the invention is designated BMS61. The nucleotide sequence of BMS61 is shown in SEQ ID NO:43. BMS61 cDNA represents a transcript of 1152 nucleotide with a translation start codon (ATG) at position 276, a translation stop codon at position 795, and a poly(A) tail at position 1150. The DNA sequence between nucleotides 276 and 794 encodes a protein of 173 amino acid residues, as shown in SEQ ID NO:44. A BMS61 polynucleotide can comprise at least 103, 200, 300, 400, 500, 600, 700, 800, 900, 1000 or 1100 contiguous nucleotides of SEQ ID NO:43, at least 11, 12, 13, 14, 15, 20, 25, 30, 35, 40, or 50 contiguous nucleotides selected from nucleotides 1-280, 270-319, 378-423, 414-492, 532-570, or 1086-1152 of SEQ ID NO:43, or the complements thereof.

The present invention provides isolated genes which comprise the coding sequences disclosed herein. The genes can be isolated in accordance with known methods using the sequence information disclosed herein. Such methods include the preparation of probes or primers from the disclosed sequence information for identification and/or amplification of genes in appropriate genomic libraries or other sources of genomic materials.

The invention also provides means of altering the expression of genes which have the coding sequences disclosed herein. In one embodiment of the invention, expression of an endogenous gene having a coding sequence as shown in SEQ ID NOS:1, 3, 5, 7, 9, 11, 13, 15, 17, 19, 21, 23, 25, 27, 29, 31, 33, 35, 37, 39, 41, or 43 in a cell can be altered by introducing in frame with the endogenous gene a DNA construct comprising a transcription unit by homologous recombination to form a homologously recombinant cell comprising the transcription unit. The transcription unit comprises a targeting sequence, a regulatory sequence, an exon, and an unpaired splice donor site. This method of affecting endogenous gene expression is taught in U.S. Pat. No. 5,641,670.

The targeting sequence is a segment of at least 10, 12, 15, 20, or 50 contiguous nucleotides selected from the nucleotide sequences shown in SEQ ID NOS:1, 3, 5, 7, 9, 11, 13, 15, 17, 19, 21, 23, 25, 27, 29, 31, 33, 35, 37, 39, 41, and 43. The transcription unit is located upstream to a coding sequence of the endogenous gene. The exogenous regulatory sequence directs transcription of the coding sequence of the gene.

In another embodiment of the invention, expression of a gene with a coding sequence as shown in SEQ ID NOS:1, 3, 5, 7, 9, 11, 13, 15, 17; 19, 21, 23, 25, 27, 29, 31, 33, 35, 37, 39, 41, or 43 is decreased using a ribozyme, an RNA molecule with catalytic activity. See, e.g., Cech, 1987, Science 236: 1532-1539; Cech, 1990, Ann. Rev. Biochem. 59:543-568; Cech, 1992, Curr: Opin: Struct. BioL 2: 605-609; Couture and Stinchcomb, 1996, Trends Genet. 12:510-515. Ribozymes can be used to inhibit gene function by cleaving an RNA sequence, as is known in the art (e.g., Haseloff et al., U.S. 5,641,673).

The coding sequences disclosed herein can be used to generate a ribozyme which will specifically bind to the corresponding mRNA. Methods of designing and constructing ribozymes which can cleave other RNA molecules in trans in a highly sequence specific manner have been developed and described in the art (see Haseloff et al., Nature 334:585-591,1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201). Longer complementary sequences can be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target.

Ribozymes can be introduced into cells as part of a DNA construct, as is known in the art. The DNA construct can also include transcriptional regulatory elements, such as a promoter element, an enhancer or UAS element, and a transcriptional terminator signal, for controlling transcription of the ribozyme in the cells.

Mechanical methods, such as microinjection, liposome-mediated transfection, electroporation, or calcium phosphate precipitation, can be used to introduce the ribozyme-containing DNA construct into cells in order to decrease gene expression.

Alternatively, if it is desired that the cells stably retain the DNA construct, it can be supplied on a plasmid and maintained as a separate element or integrated into the genome of the cells, as is known in the art.

Expression of a gene with a coding sequence as shown in SEQ ID NOS:1, 3, 5, 7, 9, 11., 13, 15, 17, 19,.21, 23, 25,.27, 29, 31, 33, 35, 37, 39, 41, or 43 can also be altered using an antisense oligonucleotide. The sequence of the antisense oligonucleotide is complementary to at least a portion of a coding sequence disclosed herein. Preferably, the antisense oligonucleotide is at least six nucleotides in length, but can be at least 8, 11, 12, 15; 20, 25, 30, 35, 40, 45, or 50 nucleotides long. Longer sequences, such as the complement of the nucleotide sequences shown in SEQ ID NOS:1, 3, 5; 7, 9,11, 13, 15, 17, 19, 21, 23, 25, 27, 29, 31, 33, 35, 37, 39, 41, or 43′, can also be used. Antisense oligonucleotides can be provided in a construct of the invention and introduced into cells using transfection techniques known in the art..

Antisense oligonucleotides can be composed of deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, 1994, Meth. Mol. Biol. 20:1-8; Sonveaux, 1994, Meth. Mol. Biol. 26:1-72; Uhlmann et al., 1990, Chem. Rev. 90:543-583.

Precise complementarity is not required for successful duplex formation between an antisense molecule and its complementary coding sequence. Antisense molecules which comprise, for example, 2, 3, 4, or 5 or more stretches of contiguous nucleotides which are precisely complementary to a coding sequence of the invention, each separated by a stretch of contiguous nucleotides which are not complementary to adjacent coding sequences, can provide targeting specificity for mRNA. Preferably, each stretch of contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular coding sequence of the invention.

Antisense oligonucleotides can be modified without affecting their ability to hybridize to a coding sequence of the invention: These modifications can be internal or at one or both ends of the antisense oligonucleotide. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, can also be employed in a modified antisense oligonucleotide. These modified oligonucleotides can be prepared by methods well known in the art. Agrawal et al., Trends Biotechnol. 10:152-158,1992; Uhlmann et al.; Chem. Rev. 90:543-584, 1990; Uhlmann et al., Tetrahedron. Lett. 215:3539-3542, 1987.

Antibodies of the invention can also be used to decrease the function of proteins of the invention. Specific antibodies bind to a protein of the invention to prevent the protein from functioning in the cell. Polynucleotides encoding single-chain antibodies of the invention can be introduced into cells using standard transfection techniques. Alternatively, therapeutic antibodies o f t he invention c an b e targeted to a particular c ell type, for example, by binding an antibody to a coupling molecule which is specific for both the antibody and the target, as disclosed in WO 95/08577. The coupling molecule can comprise immunoglobulin binding domains.

Proteins of the invention comprise the amino acid sequences shown in SEQ ID NOS:2, 4, 6, 8, 10, 12, 14, 16, 18, 20, 22, 24, 26, 28, 30, 32, 34, 36, 38, 40, 42, and 44.

Protein or polypeptide fragments which are capable of exhibiting biological activity are also encompassed by the present invention.

Non-naturally occurring protein variants which retain substantially the same biological activities as naturally occurring proteins of the invention are also included here. Preferably, naturally or non-naturally occurring protein variants have amino acid sequences which are at least 65%, 75%, 85%, 90%, or 95% identical to the amino acid sequences shown in SEQ ID NOS:2, 4, 6, 8, 10, 12, 14, 16, 18, 20, 22, 24, 26, 28, 30, 3,2, 34, 36, 38, 40, 42, and 44 are secreted proteins, and have similar biological properties. More preferably, the molecules are 98% identical. Percent identity can be determined using computer programs which use the Smith-Waterman algorithm using an affine gap search with the following parameters: a gap open penalty of 12 and a gap extension penalty of 1.

Guidance in determining which amino acid residues may be substituted, inserted, or deleted Without abolishing biological or immunological activity may be found using computer programs well known in the art, such as DNASTAR software. Preferably, amino acid changes in protein variants or derivatives are conservative amino acid changes, i.e., substitutions of similarly charged of uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serene, threonine, tyrosine) amino acids. Phenylalanine, tryptophan, and tyrosine are sometimes classified jointly as aromatic amino acids. It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting protein variant.

Variants of proteins of the invention include glycosylated forms, aggregative conjugates with other molecules, and covalent conjugates with unrelated chemical moieties. Variants of the invention also include allelic variants, species variants, and muteins. Truncations or deletions of regions which do not affect the properties or functions of proteins of the invention are also variants. Covalent variants can be prepared by linkage of functionalities to groups which are found in the amino acid chain or at the N- or C-terminal residue, as is known in the art.

The invention also provides polypeptide fragments of the disclosed secreted proteins. Polypeptides of the invention comprise less than all of the amino acid sequences shown in SEQ ID NOS:2, 4, 6, 8, 10, 12, 14, 16, 18, 20, 22, 24, 26, 28, 30, 32, 34, 36, 38, 40, or 42 in the same primary order as found in the full-length amino acid sequences. For example, polypeptides of the invention can comprise at least 95′, 100, 120, 130, or 140 contiguous amino acids of SEQ ID NO:2.

Other polypeptides of the invention can comprise at least 6, 8, 10, 20, 30, 40, 50, 60, 70, 80, 90, 100, 101, 110, 120, 130, 150, 160, 170, or 180 contiguous amino acids of SEQ ID NO:4.

Yet other polypeptides of the invention can comprise at least 14, 15, 16, 18, 20, 25, or 30 contiguous amino acids selected from amino acids 1-312 of SEQ ID NO:6 or at least 75, 100, 125, 150, 175, 179, 200, 225, 250, 275, 300, 325, or 350 contiguous amino acids of SEQ ID NO:6.

Even other polypeptides of the invention can comprise at least 17, 18, 19, 20, 25, or 30 contiguous amino acids selected from amino acids 1-287 of SEQ ID NO:8 or at least 136, 140, 150, 150, 179, 200, 250, 300, 350, or 400 contiguous amino acids selected from SEQ ID NO:8.

Still other polypeptides of the invention can comprise at least 31, 32, 35, 40, or 45 contiguous amino acids selected from amino acids 1-238 of SEQ ID NO:10 or at least 82, 85, 100,132, 150, 200, 225, 250, or 275 contiguous amino acids of SEQ ID NO:10.

Other polypeptides of the invention can comprise at least 6, 7, 8, 9, 10, 15, or 20 contiguous amino acids selected from amino acids 1-184 or 270-362 of SEQ ID NO:12, at least 8, 9, 10, 12, 15, 20, or 25 contiguous amino acids selected from amino acids 268-364 of SEQ ID NO: 12, at least 27, 30, 35, or 40 contiguous amino acids selected from amino acids 250-383 of SEQ ID NO:12, or at least 96, 100, 150, 200, 250, 300, or 350 contiguous amino acids selected from SEQ ID NO:12.

Yet other polypeptides of the invention can comprise at least 6, 7, 8, 9, 10, 12, 15, or 20 contiguous amino acids selected from amino acids 1-111 or 204-261 of SEQ ID NO:14, at least 17, 18, 20, 25, or 30 contiguous amino acids selected from amino acids 1-150 of SEQ ID NO:14, at least 75, 80, 100, 104, 125, 150, 175, 200, 225, or 250 contiguous amino acids of SEQ ID NO:14.

Even other polypeptides of the invention can comprise at least 8, 10, 12, 14, 16, 20, 30, 40, 50, 75, 100, 125, 150, 175, 200, 225, 250, 275, 300, 325, 350, 375, 400, 425, 450, 475, 500, 525, or 550 contiguous amino acids of SEQ ID NO:16.

Still other polypeptides of the invention can comprise at least 39, 40, 45, 46, or 50 contiguous amino acids selected from amino acids 13-232 of SEQ ID NO:18 or at least 46, 50, 55, 60, 75, 100, 125,150, 175, 200, or 225 contiguous amino acids of SEQ ID NO:18.

Other polypeptides of the invention can comprise at least 6; 8, 10, 12, 15, 20, 25, 30, 50, 75, 100, 125, or 140 contiguous amino acids from SEQ ID NO:20.

Yet other polypeptides of the invention can comprise at least 7, 8, 10, 12, 15, 20, 25, 30, 50, 75, 100, 125, 150, or 160 contiguous amino acids from SEQ ID NO:22.

Even other polypeptides of the invention comprise at least 7, 8, 10, 12, 15, 20, 25, 30, 50, 75, 100,.125, 150, 175, 200,225, 250, or 275 contiguous amino acids of SEQ ID NO:24.

Still other polypeptides of the invention comprise at least 11, 12, 15, 18, 20, 25, 30, 35, 50, 75, 100, 125, 150, 175, 200, or 225 contiguous amino acids of SEQ ID NO:26.

Other polypeptides of the invention comprise at least 6, 8, 10, 12, 15, or 20 contiguous amino acids selected from amino acids 1-31 of SEQ ID NO:28 or at least 257, 260, 270, 280, 290, 300, 325, 350, 375, 400, 425, or 450 contiguous amino acids of SEQ ID NO:28.

Yet other polypeptides of the invention comprise at least 6, 8, 10, 12, 15, 18, 20, 25, 30, 50, 75, 100, 125, 150, 175, or 200 contiguous amino acids of SEQ ID NO:30.

Even other polypeptides of the invention comprise at least 6, 8, 10, 12, 15, or 20 contiguous amino acids selected from amino acids 1-65 of SEQ ID NO:32 or at least 117, 120, 150, 175, 200, or 225 contiguous amino acids of SEQ ID NO:32.

Still other polypeptides of the invention comprise at least 6, 8, 10, 12, 15, 20, 25, 30, 50, 75, 100, 125, 150, or 175 contiguous amino acids of SEQ ID NO:34.

Other polypeptides of the invention comprise at least 14, 15, 18, 20, 25, 30, 50, 75, 100, 125, 150, 175, 200, 225, 250, 275, or 300 contiguous amino acids of SEQ ID NO:36.

Yet other polypeptides of the invention comprise at least 19, 20, 25, 30, 35, 40, 50, 75, 100, 125, 150, 175, 200, 224, 250, 275, 300, or 325 contiguous amino acids of SEQ ID NO:38.

Even other polypeptides of the invention comprise at least 8, 10, 12, 15, 18, 20, 25, 30, 50, 75, 100, 125, 150, 175, 200, 225; 250, 275, 300, 325, 350, 375, 400, 425, 450, 475, or 500 contiguous amino acids of SEQ ID NO:40.

Still other polypeptides of the invention comprise at least 7, 8, 10, 12, 15, 20, 30, 50, 75, 100, or 125 contiguous amino acids of SEQ ID NO:42.

Other polypeptides of the invention comprise at least 10, 12, 15, 20, 25, 30, 50, 75, 100, 125, 150, or 170 contiguous amino acids of SEQ ID NO:44.

Polypeptides can be linear or can be cyclized using known methods; for example, as described in Saragovi et al:, Bio/Technology 10, 773-778 (1992) or McDowell et al., J. Amer. Chem. Soc. 114, 9245-9253 (1992). Polypeptides can optionally be fused to carrier molecules such as immunoglobulins and used, for example, to increase the number of protein binding sites in a molecule or a molecular complex. Polypeptide fragments of the protein can be fused through linker sequences to the Fc portion of an immunoglobulin. Fusion of polypeptide fragments to the Fc portions of an IgG molecule can provide a bivalent form of a protein. Other immunoglobulin Fc portions, for example, IgM or IgA, can be used to provide multivalent forms of a protein.

Receptors or other membrane-bound proteins of the invention can be solubilized by deleting part of all of the intracellular and transmembrane domains of the protein, such that the protein can be fully secreted from a cell in which it is expressed. Intracellular and transmembrane domains of proteins of the invention can be identified using known techniques for determination of such domains from sequence information.

The invention also provides species homologs of the disclosed polynucleotides and proteins. Species homologs can be isolated and identified, for example, by making suitable probes or primers from the sequences disclosed herein and screening a suitable nucleic acid source from the desired species. The invention also encompasses allelic variants of the disclosed polynucleotides or proteins. Allelic variants are naturally-occurring alternative forms of polynucleotides which encode proteins which are identical, homologous, or related to those encoded by the polynucleotides shown in SEQ ID NOS:1, 3, 5, 7, 9, 11, 13, 15, 17, 19, 21, 23, 25, 27, 29, 31, 33, 35, 37, 39, 41, and 43.

Proteins of the invention can be prepared by culturing transformed host cells under culture conditions suitable for expression of the recombinant protein. If a protein of the invention is produced in a yeast or bacterial expression system, it may be necessary to modify the protein, for example, by phosphorylation or glycosylation of appropriate sites, in order to obtain the protein in a functional form. Such covalent attachments can be made using known chemical or enzymatic methods. The resulting expressed protein can then be purified from the culture (i.e., from culture medium or cell extracts) using known purification—techniques, such as size exclusion chromatography, ammonium sulfate fractionation, ion exchange chromatography, affinity chromatography, crystallization, electrofocusing, immunoprecipitation, immunoaffinity chromatography, and preparative gel electrophoresis:

A protein of the invention can optionally be expressed in a form which will facilitate purification. A protein can be expressed as a fusion protein with,, for example, maltose binding protein (MBP), glutathione-S-transferase (GST), or thioredoxin (TRX). Kits for expression and purification of such fusion proteins are commercially available from New England BioLab (Beverly, Mass.), Pharmacia (Piscataway, N.J.) and In Vitrogen, respectively. Alternatively, a protein of the invention can be tagged with an epitope and subsequently purified using a specific antibody directed to the epitope. One such epitope, Flag, is commercially available from Kodak (New Haven, Conn.).

A protein of the invention can be expressed as a product of transgenic animals, e.g., as a component of the milk of transgenic cows, goats, pigs, or sheep which are characterized by somatic or germ cells containing a nucleotide sequence encoding the protein. Proteins of the invention can also be produced by known conventional chemical synthesis. Methods for constructing the proteins of the present invention by synthetic means, such as solid phase peptide synthesis, are well known in the art.

Fusion proteins comprising amino acid sequences of proteins of the invention can also be constructed. Fusion proteins are useful for generating antibodies against amino acid sequences and for use in various assay systems. For example, fusion proteins can be used to identify proteins which interact with proteins of the invention. Physical methods, such as protein affinity chromatography, or library-based assays for protein-protein interactions such as the yeast two-hybrid or phage display systems, can also be used for this purpose. Such methods are well known in the art and can also be used as drug screens.

A fusion protein of the invention comprises two protein segments fused together by means of a peptide bond. The first protein segment consists of at least 95, 100, 120, 130, or 140 contiguous amino acids of SEQ ID NO:2, at least 6, 8, 10, 20, 30; 40, 50, 60, 70, 80, 90; 100, 101, 110, 120, 130, 150, 160, 170, or 180 contiguous amino acids of SEQ ID NO:4, at least 14, 15, 16, 18, 20, 25, or 30 contiguous amino acids selected from amino acids 1-312 of SEQ ID NO:6 or at least 75, 100; 125, 150; 175, 179, 200, 225, 250, 275, 300, 325, or 350 contiguous amino acids of SEQ ID NO:6, at least 17, 18, 19, 20, 25, or 30 contiguous amino acids selected from amino acids 1-287 of SEQ ID NO:8 or at least 136, 140, 150, 150, 179, 200, 250, 300, 350, or 400 contiguous amino acids selected from SEQ ID NO:8, at least 31, 32; 35, 40, or 45 contiguous amino acids selected from amino acids 1-238 of SEQ ID NO:10, or at least 82, 85, 100, 132, 150, 200, 225, 250, or 275 contiguous amino acids of SEQ ID NO:10, at least 6, 7, 8, 9, 10, 15, or 20 contiguous amino acids selected from amino acids 1-184 or 270-362 of SEQ ID NO:12, at least 8, 9, 10, 12, 15, 20, or 25 contiguous amino acids selected from amino acids 268-364 of SEQ ID NO:12, at least 27, 30, 35, or 40 contiguous amino acids selected from amino acids 250-383 of SEQ ID 140:12, or at least 96, 100, 150, 200, 250, 300, or 350 contiguous amino acids selected from SEQ ID 140:12, at least 6, 7, 8, 9, 10, 12, 15, or 20 contiguous amino acids selected from amino acids 1-111 or 204-261 of SEQ ID NO:14, at least 17, 18, 20, 25, or 30 contiguous amino acids selected from amino acids 1-150 of SEQ ID 140:14, at least 75, 80, 100, 104, 125, 150, 175, 200, 225, or 250 contiguous amino acids of SEQ ID 140:14, at least 8, 10, 12, 14, 16, 20, 30, 40, 50, 75, 100, 125, 150, 175, 200, 225, 250, 275, 300, 325, 350, 375, 400, 425, 450, 475, 500, 525, or 550 contiguous amino acids of SEQ ID 140:16, at least 39, 40, 45, 46, or 50 contiguous amino acids selected from amino acids 13-232 of SEQ ID 140:18 or at least 46, 50, 55, 60, 75, 100, 125, 150, 175, 200, or 225 contiguous amino acids of SEQ ID 140:18, at least 6, 8, 10, 12, 15, 20, 25, 30, 50, 75, 100, 125, or 140 contiguous amino acids from SEQ ID 140:20, at least 7, 8, 10, 12, 15, 20, 25, 30, 50, 75, 100, 125, 150, or 160 contiguous amino acids from SEQ ID 140:22, at least 7, 8, 10, 12, 15, 20, 25, 30, 50, 75, 100, 125, 150, 175, 200, 225; 250, or 275 contiguous amino acids of SEQ ID 140:24, at least 11, 12, 15, 18, 20, 25, 30, 35, 50, 75, 100, 125, 150, 175, 200, or 225 contiguous amino acids of SEQ ID 140:26, at least 6, 8, 10,.12, 15, or 20 contiguous amino acids selected from amino acids 1-31 of SEQ ID 140:28 or at least 257, 260, 270, 280, 290, 300, 325, 350, 375, 400, 425, or 450 contiguous amino acids of SEQ ID 140:28, at least 6; 8, 10, 12, 15, 18, 20, 25, 30, 50, 75, 100, 125, 150, 175, or 200 contiguous amino acids of SEQ ID NO:30, at least 6, 8., 10, 12, 15, or 20 contiguous amino acids selected from amino acids 1-65 of SEQ ID 140:32 or at least 117, 120, 150, 175, 200, or 225 contiguous amino acids of SEQ ID 140:32, at least 6, 8, 10, 12; 15, 20, 25, 30, 50, 75, 100, 125, 150, or 175 contiguous amino acids of SEQ ID NO:34, at least 14, 15, 18, 20, 25, 30, 50; 75, 100, 125, 150, 175, 200, 225, 250, 275; or 300 contiguous amino acids of SEQ ID 140:36, at least 19, 20, 25, 30, 35, 40, 50, 75, 100, 125, 150; 175, 200, 224, 250, 275, 300, or 325 contiguous amino acids of SEQ ID 140:38, at least 8, 10, 12, 15, 18, 20, 25, 30, 50, 75, 100, 1.25, 150, 175, 200, 225, 250, 275, 300, 325, 350, 375, 400, 425, 450, 475, or 500 contiguous amino acids of SEQ ID 140:40, at least 7, 8, 10, 12, 15, 20, 30, 50, 75, 100, or 125 contiguous amino acids of SEQ ID NO:42, at least 10, 12, 15, 20, 25, 30, 50, 75, 100, 125, 150, or 170 contiguous amino acids of SEQ ID NO:44. The amino acids can also be selected from biologically active variants of those sequences. The first protein segment can also be a full-length protein as shown in SEQ ID NOS:2, 4, 6, 8, 10, 12, 14, 16, 18, 20, 22, 24, 26, 28, 30, 32, 34, 36, 38, 40, 42, or 44. The first protein segment can be N-terminal or C-terminal, as is convenient.

The second protein segment can be a full-length protein or a protein fragment or polypeptide. Proteins commonly used in fusion protein construction include βgalactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex A DNA binding domain (DBD) fusions, GAL4 DNA binding domain fusions, and herpes simplex virus (HSV) BP 16 protein fusions.

Fusion proteins of the invention can be made by covalently linking the first and second protein segments or by standard procedures in the art of molecular biology. Recombinant DNA methods can be used to prepare fusion proteins, for example, by making a DNA construct which comprises coding sequences selected from SEQ ID NOS: 1, 3, 5, 7, 9, 11, 13, 15, 17, 19, 21, 23,,25, 27, 29, 31, 33, 35, 37, 39, 41, or 43 in proper reading frame with nucleotides encoding the second protein segment and expressing the DNA construct in a host cell, as is known in the art. Many kits for constructing fusion proteins are available from companies which supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.); and Quantum Biotechnologies (Montreal, Canada; 1-888-DNA-KITS).

Isolated proteins, polypeptides, biologically active variants, or fusion proteins can be used as immunogens, to obtain a preparation of antibodies which specifically bind to epitopes of the secreted proteins disclosed herein. The entire protein or fragments of the protein can be used as an immunogen, optionally conjugated to a hapten, such as keyhole limpet hemocyanin.

The antibodies can be used, inter alia, to detect proteins of the invention in human tissue or in fractions thereof. The antibodies can also be used to detect the presence of mutations in the genes encoding these proteins which result in under- or over-expression of proteins of the invention or in expression of a secreted protein with altered size or electrophoretic mobility. By binding to a protein of the invention, antibodies can also alter the functions of the protein.

Antibodies which specifically bind to a protein of the invention can be useful diagnostic agents. Antibodies can also be used to treat conditions associated with the protein, including forms of cancer in which abnormal expression of the protein is involved. In the case of neoplastic cells, antibodies which specifically bind to the protein can be useful for suppressing the metastatic spread of the neoplastic cells, which can be mediated by the protein.

Antibodies which specifically bind to epitopes of the secreted proteins, polypeptides, fusion proteins, or biologically active variants disclosed herein can be used in immunochemical assays, including but not limited to Western blots, ELISAs, radioimmunoassays, immunohistochemical assays, immunoprecipitations, or other immunochemical assays known in the art. Typically, antibodies of the invention provide a detection signal at least 5-, 10-, or 20-fold higher than a detection signal provided with other proteins when used in such immunochemical assays. Preferably, antibodies which specifically bind to epitopes of a particular secreted protein do not detect other proteins in immunochemical assays and can immunoprecipitate that protein or polypeptide fragments of the protein from solution.

Specific antibodies specifically bind to epitopes present in a secreted protein having one of the amino acid sequences shown in SEQ ID NOS:2; 4, 6, 8, 10, 12, 14, 16, 18, 20, 22, 24, 26,,28, 30, 32, 34, 36; 38, 40, 42; or 44 or to biologically active variants of those sequences. Typically; at least 6, 8, 10, or 12 contiguous amino acids are required to form an epitope. However, epitopes which involve non-contiguous amino acids may require more, e.g., at least 15, 25, or 50 amino acids. Preferably, the epitopes are not present in other human proteins.

Epitopes of proteins of the invention which are particularly antigenic can be selected, for example, by routine screening of polypeptide fragments of the protein for antigenicity or by applying a theoretical method for selecting antigenic regions of a protein to the amino acid sequences disclosed herein. Such methods are taught, for example, in Hopp and Wood, Proc. Natl. Acad. Sci. U.S.A. 78, 3824-28 (1981), Hopp and Wood, Mol. Immunol. 20, 483-89 (1983), and Sutcliffe et al., Science 219, 660-66 (1983).

Any type of antibody known in the art can be generated to bind specifically to epitopes of a secreted protein of the invention. For example, preparations of polyclonal and monoclonal antibodies can be made using standard methods which are well known in the art. Similarly, single-chain antibodies can also be prepared. Single-chain antibodies can be isolated, for example, from single-chain immunoglobulin display libraries, as is known in the art. The library is “panned” against amino acid sequences of a particular protein of the invention, and a number of single chain antibodies which bind with high-affinity to different epitopes of the protein can be isolated. Hayashi et al., 1995, Gene 160:129-30. Single-chain antibodies can also be constructed using a DNA amplification method, such as the polymerase chain reaction (PCR), using hybridoma cDNA as a template. Thirion et al., 1996, Eur. J. Cancer Prev. 5:507-11.

Single-chain antibodies can be mono- or bispecific, and can be bivalent or tetravalent. Construction of tetravalent, bispecific single-chain antibodies is taught, for example, in Coloma and Morrison, 1997, Nat. Biotechnol. 15:159-63. Construction of bivalent, bispecific single-chain antibodies is taught inter alia in Mallender and Voss, 1994, J. Biol. Chem. 269:199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology. Verhaar et al., 1995, Int. J. Cancer 61:497-501; Nicholls et al., 1993,.) Immunol. Meth. 165:81-91.

Monoclonal and other antibodies can also be “humanized” in order to prevent a patient from mounting an immune response against the antibody when it is used therapeutically. Such antibodies may be sufficiently similar in sequence to human antibodies to be used directly in therapy or may require alteration of a few key residues. Sequence differences between, for example, rodent antibodies and human sequences can be minimized by replacing residues which differ from those in the human sequences, for example, by site directed mutagenesis of individual residues, or by grafting of entire complementarity determining regions. Alternatively, one can produce humanized antibodies using recombinant methods, as described in GB2188638B. Antibodies which specifically bind to epitopes of a protein of the invention can contain antigen binding sites which are either partially or fully humanized, as disclosed in U.S. Pat. No. 5,565,332.

Other types of antibodies can be constructed and used in methods of the invention. For example, chimeric antibodies can be constructed as disclosed, for example, in WO 93/03151. Binding proteins which are derived from immunoglobulins and which are multivalent and multispecific, such as the “diabodies” described in WO 94/13804, can also be prepared.

Antibodies of the invention can be purified by methods well known in the art. For example, antibodies can be affinity purified by passing the antibodies over a column to which a protein, polypeptide, biologically active variant, or fusion protein of the invention is bound. The bound antibodies can then be eluted from the column, using a buffer with a high salt concentration.

Specific-binding polypeptides other than antibodies can also be generated. Specific-binding polypeptides are polypeptides which bind with a secreted protein or its variants and which have a measurably higher binding affinity for that protein and polypeptide fragments or variants of the protein than for other polypeptides tested for binding. Higher affinity by a factor of 10 is preferred, more preferably a factor of 100. Such polypeptides can be found, for example, using the yeast two-hybrid system.

Polynucleotides and proteins of the present invention exhibit one or more of the utilities or biological activities which are identified below. Biological activities and utilities of proteins of the invention can be provided by administration or use of the proteins themselves or by administration or use of polynucleotides encoding the proteins.

A protein of the invention can exhibit cytokine, cell proliferation (either inducing or inhibiting), or cell differentiation (either inducing or inhibiting) activity, or can induce production of other cytokines in certain cell populations. Many protein factors discovered to date, including all known cytokines, have exhibited activity in one or more factor-dependent cell proliferation assays; hence, the assays serve as a convenient confirmation of cytokine activity. The activity of a protein of the invention can be evidenced by any one of a number of routine factor-dependent cell proliferation assays for cell lines including, 32D (a mouse IL-3-dependent lymphoblast cell line, ATCC No. CRL-1 1346), DA2, DAIG, TIO (a human myeloma cell line, ATCC No. CRL-9068), B9, B9/11, BaF3, MC9/G, M+(preB M+), 2E8 (a mouse IL-7-dependent lymphoblast cell line, ATCC No. TIB-239), RB5, DA1, 123, T1165, HT2 (a mouse lymphoma cell line, ATCC No. CRL-8629), CTLL2, TF-1 (a human IL-5-unresponsive lymphoblast cell line, ATCC No. CRL-2003), Mole, and CMK.

Assays for T-cell or thymocyte proliferation include those described in CURRENT PROTOCOLS IN IMMUNOLOGY, Coligan et al., eds., Greene Publishing Associates and Wiley-Interscience (particularly chapter 3, In Vitro Assays for Mouse Lymphocyte Function 3.1-3.19; and chapter 7, Immunologic Studies in Humans); Takai et al., J. Immunol. 137:3494-3500,1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; and Bowman et al:, J. Immunol. 152:1756-1761, 1994.

Assays for cytokine production and/or proliferation of spleen cells, lymph node cells, or thymocytes include those described in Kruisbeek and Shevach, Polyclonal T Cell Stimulation, in CURRENT PROTOCOLS IN IMMUNOLOGY, Vol. 1, pp. 3.12.1-3.12.14, and Schreiber, Measurement of Mouse, and Human Interleukin Gamma, in CURRENT PROTOCOLS IN IMMUNOLOGY, Vol. 1, pp. 6.8.1-6.8.8.

Assays for proliferation and differentiation of hematopoietic and lymphopoietic cells include those described in Bottomly, Measurement of Human and Murine Interleukin 2 and Interleukin 4, in CURRENT PROTOCOLS IN IMMUNOLOGY, vol. 1, pp. 6.3.1-6.3.12; deVries et al., J. Exp. Med. 173:1205-121 1, 1991; Moreau et al., Nature 336:690-692, 1988; Greenberger et al., Proc. Natl. Acad. Sci. USA. $0:2931-2938, 1983; Nordan, R., Measurement of mouse and human interleukin 6, in CURRENT PROTOCOLS IN IMMUNOLOGY, Vol. 1, pp. 6.6.1-6.6.5; Smith et al., Proc. Natl. Acad. Sci. U.S.A. 83:1857-1861, 1986; Bennett et al., Measurement of Human Interleukin 11, in CURRENT PROTOCOLS IN IMUNOLOGY, Vol. 1, pp. 6.15.1; Ciarletta et al., Measurement of mouse and human Interleukin 9, in CURRENT PROTOCOLS IN IMMUNOLOGY, Vol. 1, p. 6.13.1.

Assays for T cell clone responses to antigens (which will identify, among others, proteins that affect APC-T cell interactions as well as direct T cell effects by measuring proliferation and cytokine production) include those described in CURRENT PROTOCOLS IN IMMUNOLOGY, especially chapters 3 (In Vitro Assays for Mouse Lymphocyte Function), chapter 6 (Cytokines and Their Cellular Receptors), and chapter 7 (Immunologic Studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; and Takai et al., J. Immunol. 140:508-512, 1988.

A protein of the present invention can be useful to support colony forming cells or factor-dependent cell lines, to regulate hematopoiesis, and to treat myeloid or lymphoid cell deficiencies. Such proteins can be used, either alone or in combination with other cytokines, to support the growth and proliferation of erythroid progenitor cells. The proteins can also be used to treat various anemias, in conjunction with irradiation or chemotherapy to stimulate the production of erythroid precursors or erythroid cells.

A protein of the invention can have CSF activity and can be used to support the growth and proliferation of myeloid cells, such as granulocytes, monocytes, or macrophages. Proteins with such activity can be used, for example; in conjunction with chemotherapy to prevent or treat myelo-suppression. Proteins of the invention can also be used to support the growth and proliferation of megakaryocytes and platelets, thereby allowing prevention-or treatment of platelet disorders such as thrombocytopenia Proteins with such activity can be used to support the growth and proliferation of hematopoietic stem cells, either in place of or in conjunction with platelet transfusions. Proteins of the invention can be used to treat stem cell disorders, such as aplastic anemia and paroxysmal nocturnal hemoglobinuria, or to repopulate the stem cell compartment after irradiation or chemotherapy, either in-vivo or ex-vivo. For example, a protein of the invention can be used in conjunction with homologous or heterologous bone marrow transplantation or peripheral progenitor cell transplantation.

Suitable assays for proliferation and differentiation of various hematopoietic lines are cited above. Assays for embryonic stem cell differentiation which can identify proteins which influence embryonic hematopoiesis include those described in Johansson et al. Cellular Biology 15:141-151, 1995; Keller et al., Molecular and Cellular Biology 13:473-486, 1993; and McClanahan et al., Blood 81:2903-2915, 1993.

Assays for stem cell survival and differentiation include those described in Freshney, Methylcellulose colony forming assays, in CULTURE OF HEMATOPOIETIC CELLS, Freshney et al. eds., pp. 265-268, Wiley-Liss, Inc., New York, N.Y. 1994; Hirayama et al., Proc. Natl. Acad. Sci. USA 89:5907-5911, 1992; McNiece and Briddell, Primitive hematopoietic colonyforming cells with high proliferative potential, in CULTURE OF HEMATOPOIETIC CELLS, pp. 23-39; Neben et al., Experimental Hematology 22:353-359, 1994; Ploemacher, Cobblestone area forming cell assay, in CULTURE OF HEMATOPOIETIC CELLS, pp. 1-21; Spooncer et al., Long term bone marrow cultures in the presence of stromal cells, in CULTURE OF HEMATOPOIETIC CELLS, pp. 163-179; Sutherland, Long term culture initiating cell assay, in CULTURE OF HEMATOPOIETIC CELLS, pp. 139-i62. Such assays can be used to identify proteins which regulate lympho-hematopoiesis.

Compositions of the invention relate to isolated (purified) polypeptides and polynucleotides. These compositions are substantially free of other human proteins or human polynucleotides. A composition containing A is “substantially free of B when at least 85% .by weight of the total A+B in the composition is A. Preferably, A comprises at least about 90% by weight of the total of A+B in the composition, more preferably at least about 96% or even 99% by weight.

A protein of the invention also can have utility in compositions used for growth or differentiation of bone; cartilage, tendon, ligament, or nerve tissue, as well as for wound healing and tissue repair and replacement, and in the treatment of bums, incisions, and ulcers.

Proteins of the present invention can induce cartilage and/or bone growth in circumstances where bone is not normally formed and thus have an application in healing bone fractures and cartilage damage or defects in humans and other animals. A preparation employing a protein of the invention can have prophylactic use in closed as well as open fracture reduction and also in the improved fixation of artificial joints. De novo bone formation induced by an osteogenic agent contributes to the repair of congenital, trauma- or surgery-induced craniofacial defects and also is useful in cosmetic plastic surgery.

A protein of this invention can also be used in the treatment of periodontal disease and in other tooth repair processes. Such agents can provide an environment to attract bone-forming cells, stimulate growth of bone-forming cells, or induce differentiation of progenitors of bone-forming cells. A protein of the invention can be used to treat osteoporosis or osteoarthritis, for example, through stimulation of bone and/or cartilage repair or by blocking inflammation. Mechanisms of destroying tissue mediated by inflammatory processes, such as collagenase or osteoclast activity, can also be inhibited.

Tendon or ligament formation can also be influenced by a protein of the invention. A protein of the invention which induces tendon/ligament-like tissue of other tissue formation in circumstances where such tissue is not normally formed can be used to heal tendon or ligament tears, deformities, and other tendon or ligament defects in humans and other animals. A preparation employing a tendon/ligament-like tissue inducing protein can be used to prevent damage to tendon or ligament tissue, as ,well as in the improved fixation of tendon or ligament to bone or other tissues, and to repair defects to tendon or ligament tissue. De novo tendon/ligament-like tissue formation induced by a composition of the invention contributes to the repair of congenital, trauma-induced, or other tendon or ligament defects of other origin and can also be used in cosmetic plastic surgery, for attachment or repair of tendons or ligaments.

Compositions of the invention can provide an environment which will attract tendon- or ligament-forming cells, stimulate growth of tendon- or ligament-forming cells, induce differentiation of progenitors of tendon- or ligament-forming cells, or induce growth of tendon/ligament cells or progenitors ex vivo. Such cells can then be returned to the body to effect tissue repair. Compositions of the invention can also be used to treat tendinitis, carpal tunnel syndrome, and other tendon or ligament defects. Such compositions can optionally include an appropriate matrix and/or sequestering agent as a pharmaceutically acceptable carrier, as is well known in the art.

A protein of the invention can also be useful for proliferation of neural cells and for regeneration of nerve and brain tissue, i.e. for the treatment of central and peripheral nervous system diseases and neuropathies, as well as mechanical and traumatic disorders. More specifically, a protein can be used in the treatment of diseases such as Alzheimer's disease, Parkinson's disease, Huntington's disease, amyotrophic lateral sclerosis, and Shy-Drager syndrome. Other conditions which can be treated in accordance with the invention include mechanical and traumatic disorders, such as spinal cord disorders and head trauma, and cerebrovascular diseases, such as stroke. Peripheral neuropathies resulting from chemotherapy or other medical therapies can be treated using a protein of the invention.

Proteins of the invention can also be used to promote better or faster closure of non-healing wounds, including pressure ulcers, ulcers associated with vascular insufficiency, or surgical and traumatic wounds.

A protein of the invention can also affect generation or regeneration of other tissues, such as organs (including, for example, pancreas, liver, intestine, kidney, skin, endothelium), muscle (smooth, skeletal, or cardiac), and vascular (including vascular endothelium) tissue, or for promoting the growth of cells of which such tissues are comprised. Part of the desired effects can be by inhibition or modulation of fibrotic scarring to allow normal tissue to regenerate. A protein of the invention can also exhibit angiogenic activity.

A protein of the present invention can be useful for gut protection or regeneration, and for treatment of lung or liver fibrosis, reperfusion injury in various tissues, and conditions resulting from systemic cytokine damage. A protein of the invention can also be useful for promoting or inhibiting differentiation of tissues described above from precursor tissues or cells, or for inhibiting the growth of tissues described above.

Assays for tissue generation activity include those described for bone, cartilage, and tendon in WO 95/16035, for neuronal tissue in WO 95/05846, and for skin and endothelial tissue in WO 91/07491. Assays for wound healing activity include, for example, those described in Winter, EPIDERMAL WOUND HEALING, polypeptides 71-112 (Maibach and Rovee, eds.), Year Book Medical Publishers, Inc., Chicago, and Eaglstein and Mertz, J. Invest. Dermatol 71:382-84 (1978).

A protein of the present invention can also demonstrate activity as a receptor, receptor ligand, or inhibitor or agonist of a receptor/ligand interaction. Examples of such receptors and ligands include cytokine receptors and their ligands, receptor kinases and their ligands, receptor phosphatases and their ligands, receptors involved in cell-cell interactions and their ligands, including cellular adhesion molecules such as selectins, integrins, and their ligands, and receptor/ligand pairs involved in antigen presentation, antigen recognition and development of cellular and Immoral immune responses. Receptors and ligands are also useful for screening of potential peptide or small molecule inhibitors of the relevant receptor/ligand interaction. A protein of the invention, including fragments of receptors and ligands, can itself be useful as an inhibitor of receptor/ligand interactions.

Suitable assays for receptor-ligand activity include those described in CURRENT PROTOCOLS IN IMMUNOLOGY, chapter 7.28, Measurement of Cellular Adhesion under static conditions, pages 7.28.1-7.28.22, Takai et al., Proc. Natl. Acad Sci. USA 84:6864-6868, 1987, Bierer et al., J. Exp. Med. 168:1145-1156, 1988; Rosenstein et al. J. Exp. Med. 1 69:149-160 1 989; Stoltenborg et al., J. Immunol. Methods 1 75:59-68, 1994; Stitt et al., Cell 80:661-6′70, 1995.

A protein of the invention can be used in a pharmaceutical composition. Compositions comprising proteins or polynucleotides of the invention have therapeutic applications, both for human patients and veterinary patients, such as domestic animals and thoroughbred horses. Such compositions can optionally include a pharmaceutically acceptable carrier. In addition to protein and carrier, such a composition can also contain diluents, fillers, salts, buffers, stabilizers, solubilizers, and other materials well known in the art. Characteristics of a carrier will depend on the route of administration. Compositions of the invention can also contain cytokines, lymphokines, or other hematopoietic factors such as M-CSF, GM-CSF, TNF, IL-1, IL-2, IL-3; IL-4, IL-5, IL-6, IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, erythropoietin, or growth factors such as epidermal growth factor (EGF), platelet derived growth factor (PDGF), transforming growth factors (TGF-α and TGF-(3), or insulin-like growth factor (IGF).

A pharmaceutical composition can also contain other agents which either enhance the activity of the protein or complement its activity or use in treatment. Such additional factors and/or agents can be included in the pharmaceutical composition to produce a synergistic effect with a protein of the invention or to minimize side effects. Conversely, a protein of the invention can be included in formulations of a particular factor, such as a cytokine, lymphokine, other hematopoietic factor, thrombolytic or anti-thrombotic factor, or anti-inflammatory agent to minimize side effects of the factor.

A protein of the present invention can be active in multimers (e.g., heterodimers or homodimers) or complexes with itself or other proteins, and compositions of the invention can comprise a protein of the invention in such a multimeric or complexed form. For example, a composition of the invention can be in the form of a complex of a protein or proteins of the invention together with protein or peptide antigens. The protein or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC proteins and structurally related proteins, including those encoded by class I and class II MHC genes on host cells, can present the peptide antigen(s) to T lymphocytes: Antigen components can also be supplied as purified MHC-peptide complexes alone or with co-stimulatory molecules which can directly signal T cells. Alternatively, antibodies able to bind surface immunoglobulin and other molecules on B cells, as well as antibodies able to bind the TCR and other molecules on T cells, can be combined with a composition of the invention.

A composition of the invention can be in the form of a liposome in which a protein of the invention is combined, in addition to other pharmaceutically acceptable carriers, with amphipathic agents such as lipids, which exist in aggregated form as micelles; insoluble monolayers, liquid crystals, or lamellar layers inaqueous solution.

Suitable lipids for liposomal formulation include, monoglycerides, diglycerides, sulfatides, lysolecithin, phospholipids, saponin, bile acids, and the like. Preparation of liposomal formulations is within the level of skill in the art, as disclosed, for example, in U.S. Pat. No. 4,235,871, U.S. Pat. No. 4,501,728, U.S. Pat. No. 4,837,028, and U.S. Pat. No. 4,737,323.

A therapeutically effective amount of a protein of the invention is administered to a mammal having a condition to be treated. The amount of protein which is therapeutically effective is that amount of protein which is sufficient to treat, heal, prevent, or ameliorate the condition, or to increase the rate of such treatment. Proteins of the invention can be administered either alone or in combination with other therapeutic agents, such as cytokines, lymphokines, or other hematopoietic factors. Other therapeutic agents can be administered simultaneously or sequentially with proteins of the invention, as determined by the attending physician.

Compositions of the invention can be inhaled, ingested, applied topically, or administered by cutaneous, subcutaneous, intraperitoneal, parenteral or intravenous injection. When a therapeutically effective amount of protein of the present invention is administered orally, protein of the present invention will be in the form of a tablet, capsule, powder, solution or elixir. When administered in tablet form, the pharmaceutical composition of the invention can additionally contain a solid carrier such as a gelatin or an adjuvant. T he tablet, capsule, and powder contain from about 595%, 25-90%, 30-80%, 40-75%, or 50%, protein of the invention by weight. When administered in liquid form, a liquid carrier such as water, petroleum, oils of animal or plant origin such as peanut oil, mineral oil, soybean oil, or sesame oil; or synthetic oils can be added. The liquid form of the composition can further contain physiological saline solution, dextrose or other saccharide solution, or glycols such as ethylene glycol, propylene glycol, or polyethylene glycol. When administered in liquid form, the pharmaceutical composition contains from about 0.5-90%, 1-80%, 5-75%, 16-65%, 2050%, 10-50%, or 25-40% by weight of protein of the invention.

When a therapeutically effective amount of protein of the present invention is administered by intravenous, cutaneous, or subcutaneous injection, a pyrogen-free, parenterally acceptable aqueous solution of the protein is preferred. The skilled artisan can readily prepare an acceptable protein solution with suitable pH, isotonicity, and stability. A solution of the composition for intravenous, cutaneous, or subcutaneous injection should also contain an isotonic vehicle, such as Sodium Chloride Injection, Ringer's Injection, Dextrose Injection, Dextrose and Sodium Chloride Injection, Lactated Ringer's Injection, or other vehicle as known in the art. Stabilizers, preservatives, buffers, antioxidants, or other additives known to those of skill in the art can also be added to the composition.

The amount of protein of the present invention in the pharmaceutical composition of the present invention will depend upon the nature and severity of the condition being treated and on the nature of prior treatments which the patient has undergone. Ultimately, the attending physician will decide the amount of protein of the present invention with which to treat each individual patient. Initially, the attending physician will administer low doses o fprotein o f the present invention and observe the patient's response. Larger doses of protein of the present invention can be administered until the optimal therapeutic effect is obtained for the patient, and at that point the dosage is not increased further. It is contemplated that the various pharmaceutical compositions used to practice the method of the present invention should contain about 0.01 pg to about 100 mg (preferably about 0:1 pg to about 10 mg, more preferably about 0.1 wg to about I mg) of protein of the present invention per kg body weight.

Duration of intravenous therapy using a composition of the invention will vary, depending on the severity of the disease being treated and the condition and potential idiosyncratic response of each individual patient. It is contemplated that the duration of each application of a composition of the invention will be in the range of 12 to 24 hours of continuous intravenous administration. Ultimately, the attending physician will decide on the appropriate duration of intravenous therapy.

A composition of the invention which is useful for bone, cartilage, tendon or ligament regeneration can be administered topically, systematically, or locally in an implant or device.. Encapsulation or injection in a viscous form for delivery to the site of bone; cartilage or tissue damage is also possible. Topical administration can be suitable for wound healing and tissue repair. Optionally, therapeutic agents other than a protein of the invention can be included in the composition, as described above.

To affect bone or cartilage formation, a composition of the invention would include a matrix capable of delivering the composition to the site of bone or cartilage damage and for providing a structure for the developing bone and cartilage. Optimally, the matrix would be capable of resorption into the body. Matrices can be formed of materials presently in use for other implanted medical applications, the choice of material being based on biocompatibility, biodegradability, mechanical properties, cosmetic appearance, and interface properties. Suitable biodegradable matrix materials include chemically defined calcium sulfate, tricalciumphosphate, hydroxyapatite, polylactic acid, polyglycolic acid, polyanhydride, bone or dermal collagen, pure proteins, and extracellular matrix components. Suitable nonbiodegradable and chemically defined matrix materials include sintered hydroxyapatite, bioglass, aluminates, or other ceramics. Individual matrix components can be modified, for example, to affect pore size, particle size, particle shape, and biodegradability. Combinations of materials can be used, as is known in the art.

Sequestering agents, such as carboxymethyl cellulose or an autologous blood clot, can be employed to prevent protein compositions from dissociating from the matrix. Sequestering agents include cellulosic materials such as alkylcelluloses (including hydroxyalkylcelluloses), including methylcellulose, ethylcellulose, hydroxyethylcellulose,.hydroxypropylcellulose, hydroxypropyl-methylcellulose, and carboxymethylcellulose, the most preferred being cationic salts of carboxymethylcellulose (CMC). Other preferred sequestering agents include hyaluronic acid, sodium alginate, polyethylene glycol, polyoxyethylene oxide, carboxyvinyl polymer and polyvinyl alcohol. The amount of sequestering agent is based on total formulation weight, such as 0.5-20% or 1-10%, and should be an amount of sequestering, agent which prevents desorbtion of the protein from the polymer matrix but which permits, progenitor cells to infiltrate the matrix, so that the protein can. assist the osteogenic activity of the progenitor cells.

The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration, and other clinical factors. The dosage can vary with the type of matrix used in the reconstitution and whether other therapeutic agents, such as growth factors, are included. Progress of the treatment can be monitored by periodic assessment of tissue/bone growth and/or repair, for example, using X-rays, histomorphometric determinations, or tetracycline labeling.

Polynucleotides of the invention can also be used for gene therapy. Polynucleotides can be introduced either in vivo or ex vivo into cells for expression in a mammalian subject. Cells can be cultured ex vivo in the presence of proteins of the invention in order to produce a desired effect on or activity in such cells. Treated cells can then be introduced in vivo for therapeutic purposes, as is known in the art. Polynucleotides of the invention can be administered by known methods of introducing polynucleotides into a cell or organism (including in the form of viral vectors or naked DNA).

Polynucleotides of the invention can also be delivered to subjects for the purpose of screening test compounds for those which are useful for enhancing transfer of polynucleotides of the invention to a cell or for enhancing subsequent biological effects of the polynucleotides within the cell. Such biological effects include hybridization to complementary mRNA and inhibition of its translation, expression of the polynucleotide to form mRNA and/or protein, and replication and integration of the polynucleotide.

Test compounds which can be screened include any substances, whether natural products or synthetic, which can be administered to the subject. Libraries or mixtures of compounds can be tested. The compounds or substances can be those for which a pharmaceutical effect is previously known or unknown. The compounds or substances can be delivered, before; after, or concomitantly with the polynucleotides. They can be administered separately or in admixture with the polynucleotides.

Integration of delivered polynucleotides can be monitored by any means known in the art. For example, Southern blotting of the delivered polynucleotides can be performed. A change in the size of the fragments of the delivered polynucleotides indicates integration. Replication of the delivered polynucleotides can be monitored inter alia by detecting incorporation of labeled nucleotides combined with hybridization to a specific nucleotide probe. Expression of a polynucleotide of the invention can be monitored by detecting production of mRNA which hybridizes to the delivered polynucleotide or by detecting protein. Proteins of the invention can be detected immunologically. Thus, delivery of polynucleotides of the invention according to the present invention provides an excellent system for screening test compounds for their ability to enhance delivery, integration, hybridization, expression, replication or integration in an animal, preferably a mammal, more preferably a human.

Polynucleotides of the invention can be used for a variety of research purposes. Any or all of these research utilities are capable of being developed into reagent grade or kit format for commercialization as research products. For example, polynucleotides can be used to express recombinant protein for analysis, characterization, or therapeutic use. Polynucleotides can also be used as markers for tissues in which the corresponding protein is preferentially expressed, either constitutively or at a particular stage of tissue differentiation or development or in disease states. Polynucleotides can also be used as molecular weight markers on Southern gels or, when labeled, for example, with a fluorescent tag or a radiolabel, polynucleotides can be used as chromosome markers, to identify chromosomes for gene mapping. Potential genetic disorders can be identified by comparing the sequences of wild-type polynucleotides of the invention with endogenous nucleotide sequences in patients. Polynucleotides of the invention can also be used as probes for the discovery of novel, related. DNA sequences, to derive PCR primers for genetic fingerprinting, as probes to “subtract-out” known sequences in the process of discovering other novel polynucleotides, for selecting and making oligomers for attachment to a gene chip or other support, to raise anti-protein antibodies using DNA immunization techniques, and as antigens, to raise anti-DNA antibodies or to elicit another immune response.

Where the polynucleotide encodes a protein which binds or potentially binds to another protein, such as in a receptor-ligand interaction, the polynucleotide can also be used in interaction trap assays, such as the yeast two-hybrid assay, to identify polynucleotides encoding the protein with which binding occurs or to identify inhibitors of the binding interaction, for example in drug screening assays.

Proteins of the invention can similarly be used in assays to determine biological activity, including use in a panel of multiple proteins for high-throughput screening, to raise antibodies or to elicit another immune response, as a reagent in assays designed to quantitatively determine levels of the protein (or its receptor) in biological fluids, as markers for tissues in which the protein is preferentially expressed (either constitutively or at a particular stage of tissue differentiation or development or in a disease state), and to identify related receptors or ligands. Where the protein binds or potentially binds to another protein such as, for example, in a receptor-ligand interaction, the protein can be used to identify the other protein with which binding occurs or to identify inhibitors of the binding interaction. Proteins involved in these binding interactions can also be used to screen for peptide or small molecule inhibitors or agonists of the binding interaction.

Polynucleotides of the invention can also be used on polynucleotide arrays. Polynucleotide arrays provide a high throughput technique that can assay a large number of polynucleotide sequences in a sample. This technology can be used as a diagnostic tool and as a tool to test for differential expression of genes having the coding sequences disclosed herein.

To create arrays, single-stranded polynucleotide probes can be spotted onto a substrate in a two-dimensional matrix or array. The single-stranded polynucleotide probes can comprise at least 6, 7, 8, 9, 10, 11, 12, 13, 14, 15, 16, 17, 18, 19, 20, 25, or 30 or more contiguous nucleotides selected from the nucleotide sequences shown in SEQ ID NOS:1, 3, 5, 7; 9, 11, 13, 15, 17, 19, 21, 23, 25, 27, 29, 31, 33, 35, 37, 39, 41, or 43. The substrate can be any substrate to which polynucleotide probes can be attached, including but not limited to glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556;752; PCT No. WO 95/22058; and U.S. Pat. No. 5,631,734. Commercially available polynucleotide arrays, such as Affymetrix GeneChipTM, can also be used. Use of the GeneChipTM to detect gene expression is described, for example, in Lockhart et al., Nature Biotechnology 14:1675 (1996); Chee et al., Science 274:610 (1996); Hacia et aL, Nature Genetics 14:441, 1996; and Kozal et al., Nature Medicine 2:753, 1996.

Biological samples comprising single-stranded polynucleotides can be labeled and then hybridized to the probes. Detectable labels which can be used include but are not limited to radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Biological samples in which expression of genes comprising polynucleotides of the invention can be examined include samples of diseased and nondiseased tissues, samples of tissues suspected of being diseased (particularly tissues suspected of being neoplastic), samples of different cell types, samples of cells at different developmental stages, samples of tissues from different species, and the like.

The complete contents of all references cited in this disclosure are expressly incorporated herein by reference. While certain embodiments of the invention have been described with particularity herein, those of skill in the art will recognize that various modifications of the invention can be made. It is understood that such modifications and variations are included within the scope of the appended claims.

1. An isolated and purified protein comprising an amino acid sequence which is at least 85% identical to an amino acid sequence selected from the group consisting of SEQ ID NOS:2, 4, 6, 8, 10, 12, 14, 16, 18, 20, 22, 24, 26, 28, 30, 32, 34, 36, 38, 40, 42, and 44, wherein percent identity is determined using a Smith-Waterman homology search algorithm using an affine gap search with a gap open penalty of 12 and a gap extension penalty of 1.

2. The isolated and purified protein of claim 1 wherein the amino acid sequence comprises an amino acid sequence selected from the group consisting of SEQ ID NOS:2, 4, 6, 8, 10, 12, 14, 16, 18, 20, 22, 24, 26, 28, 30, 32, 34, 36, 38, 40, 42, and 44.

3. An isolated and purified protein comprising an amino acid sequence selected from the group consisting of at least 95 contiguous amino acids of SEQ ID 140:2, at least 101 contiguous amino acids of SEQ ID 140:4, at least 14 contiguous amino acids selected from amino acids 1-312 of SEQ ID 140:6, at least 179 contiguous amino acids of SEQ ID 140:6, at least 75 contiguous amino acids of SEQ ID 140:6, at least 179 contiguous amino acids of SEQ ID NO:8, at least 136 contiguous amino acids of SEQ ID 140:8, at least 17 contiguous amino acids selected from amino acids 1-287 of SEQ ID 14.0:8, at least 82 contiguous amino acids of SEQ ID NO:10, at least 31 contiguous amino acids selected from amino acids I-238 of SEQ ID NO:10, at least 96 contiguous amino acids of SEQ ID 140:12, at least 27 contiguous amino acids selected from amino acids 250-383 of SEQ ID 140:12, at least 6 contiguous amino acids selected from amino acids 1-184 of SEQ ID 140:12, at least 8 contiguous amino acids selected from amino acids 268-364 of SEQ ID 140:12, at least 104 contiguous amino acids of SEQ ID 140:14, at least 75 contiguous amino acids of SEQ ID 140:14, at least 17 contiguous amino acids selected from amino acids 1-150 of SEQ ID 140:14, at least 6 contiguous amino acids selected from amino acids 204-261 of SEQ ID 140:14, at least 6 contiguous amino acids selected from amino acids I-I 11 of SEQ ID 140:14, at least 8 contiguous amino acids of SEQ ID 140:16, at least 46 contiguous amino acids of SEQ ID NO:18, at least 39 contiguous amino acids selected from amino acids 13-232 of SEQ ID 140:18, at least 6 contiguous amino acids of SEQ ID 140:20, at least 7 contiguous amino acids of SEQ ID 140:22, at least 7 contiguous amino acids of SEQ ID 140:24, at least 11 contiguous amino acids of SEQ ID 140:26, at least 257 contiguous amino acids of SEQ ID NO:28, at least 6 contiguous amino acids selected from amino acids 1-31 of SEQ ID NO:28, at least 6 contiguous amino acids of SEQ ID NO:30, at least 117 contiguous amino acids of SEQ ID NO:32, at least 6 contiguous amino acids selected from amino acids 1-65 of SEQ ID NO:32, at least 6 contiguous amino acids of SEQ ID NO:34, at least 14 contiguous amino acids of SEQ ID NO:36, at least 19 contiguous amino acids of SEQ ID NO:38, at least 8 contiguous amino acids of SEQ ID NO:40, at least 7 contiguous amino acids of SEQ ID NO:42, and at least 10 contiguous amino acids of SEQ ID NO:44.

4. A fusion protein comprising two protein segments joined together with a peptide bond, wherein the first protein segment consists of an amino acid sequence selected from the group consisting of at least 95 contiguous amino acids of SEQ ID NO:2, at least 101 contiguous amino acids of SEQ ID NO:4, at least 14 contiguous amino acids selected from amino acids 1-312 of SEQ ID NO:6, at least 179 contiguous amino acids of SEQ ID NO:6, at least 75 contiguous amino acids of SEQ ID NO:6, at 1 least 179 contiguous amino acids of SEQ ID NO:8, at least 136 contiguous amino acids of SEQ ID NO:8, at least 17 contiguous amino acids selected from amino acids 1-287 of SEQ ID NO:8, at least 82 contiguous amino acids of SEQ ID NO:10, at least 31 contiguous amino acids selected from amino acids 1-238 of SEQ ID NO:10, at (cast 96 contiguous amino acids of SEQ ID NO: 12, at least 27 contiguous amino acids selected from amino acids 250-383 of SEQ ID NO:12, at least 6 contiguous, amino acids selected from amino acids 1-184 of SEQ ID NO:12, at least 8 contiguous amino acids selected from amino acids 268-364 of SEQ ID NO:12, at least 104 contiguous amino acids of SEQ ID NO: 14, at least 75 contiguous amino acids of SEQ ID NO: 14, at least 17 contiguous amino acids selected from amino acids 1-150 of SEQ ID NO:14, at least 6 contiguous amino acids selected from amino acids 204-261 of SEQ ID NO:14, at least 6 contiguous amino acids selected from amino acids 1-11 I of SEQ ID NO:14, at least 8 contiguous amino acids of SEQ ID NO:16, at least 46 contiguous amino acids of SEQ ID N.0:18, at least 39 contiguous amino acids selected from amino acids 13-232 of SEQ ID NO:18, at least 6 contiguous amino acids of SEQ ID NO:20, at least 7 contiguous amino acids of SEQ ID NO:22, at least 7 contiguous amino acids of SEQ ID NO:24, at least 11 contiguous amino acids of SEQ ID NO:26, at least 257 contiguous amino acids of SEQ ID NO:28, at least 6 contiguous amino acids selected from amino acids 1-31 of SEQ ID NO:28, at least 6 contiguous amino acids of SEQ ID NO:30, at least 117 contiguous amino acids of SEQ ID NO:32, at least 6 contiguous amino acids selected from amino acids 1-65 of SEQ ID NO:32, at least 6 contiguous amino acids of SEQ ID NO:34, at least 14 contiguous amino acids of SEQ ID NO:36, at least 19 contiguous amino acids of SEQ ID NO:38, at least 8 contiguous amino acids of SEQ ID NO:40, at least 7 contiguous amino acids of SEQ ID NO:42, and at least 10 contiguous amino acids of SEQ ID NO:44.

5. A preparation of antibodies which specifically binds to a protein comprising an amino acid sequence selected from the group consisting of SEQ ID NOS:2, 4, 6, 8, 10, 12, 14, 16, 18, 20, 22, 24, 26, 28, 30, 32, 34, 36, 38, 40, 42, and 44.

6. An isolated and purified subgenomic polynucleotide which encodes a protein comprising an amino acid sequence which is at least 85% identical to an amino acid sequence selected from the group consisting of SEQ ID NOS:2, 4, 6, 8, 10, 12, 14, 16, 18, 20, 22, 24, 26, 28, 30, 32, 34, 36, 38, 40, 42, and 44, wherein percent identity is determined using a Smith-Waterman homology search algorithm using an affine gap search with a gap open penalty of 12 and a gap extension penalty of 1.

7. The isolated and purified subgenomic polynucleotide of claim 6 wherein the amino acid sequence is selected from the group consisting of SEQ ID NOS:2, 4., 6, 8, 10, 12, 14, 16, 18, 20, 22, 24, 26, 28, 30, 32, 34, 36, 38, 40, 42, and 44.

8. An isolated and purified subgenomic polynucleotide comprising a nucleotide sequence which is at least 85% identical to a nucleotide sequence selected from the group consisting of SEQ ID NOS: 1, 3, 5, 7, 9, 11, 13, 15, 17, 19, 21, 23,25, 27, 31, 33, 35, 37, 39, 41, 43, 45; and the complements thereof, wherein percent identity is determined using a Smith-Waterman homology search algorithm using an affine gap search with a gap open penalty of 12 and a gap extension penalty of 1.

9. An isolated and purified subgenomic polynucleotide which encodes an amino acid sequence selected from the group consisting of at least 95 contiguous amino acids of SEQ ID NO:2, at least 101 contiguous amino acids of SEQ ID NO A, at least 14 contiguous amino acids selected from amino acids 1-312 of SEQ ID NO:6, at least 179 contiguous amino acids of SEQ ID NO:6, at least 75 contiguous amino acids of SEQ ID NO:6, at least 179 contiguous amino acids of SEQ ID NO:8, at least 136 contiguous amino acids of SEQ ID NO:8, at least 17 contiguous amino acids selected from amino acids 1-287 of SEQ ID NO:8, at least 82 contiguous amino acids of SEQ ID NO:10, at least 31 contiguous amino acids selected from amino acids 1-238 of SEQ ID NO:10, at least 96 contiguous amino acids of SEQ ID NO:12, at least 27 contiguous amino acids selected from amino acids 250-383 of SEQ ID NO: 12, at least 6 contiguous amino acids selected from amino acids 1-184 of SEQ ID NO:12, at least 8 contiguous amino acids selected from amino acids 268-364 of SEQ ID NO:12, at least 104 contiguous amino acids of SEQ ID NO:14, at least 75 contiguous amino acids of SEQ ID NO:14, at least 17 contiguous amino acids selected from amino acids 1-150 of SEQ ID NO:14, at least 6 contiguous amino acids selected from amino acids 204-261 of SEQ ID NO:14, at least 6 contiguous amino acids selected from amino acids 1-111 of SEQ ID NO: 14, at least 8 contiguous amino acids of SEQ ID NO:16, at least 46 contiguous amino acids of SEQ ID NO:18, at least 39 contiguous amino acids selected from amino acids 13-232 of SEQ ID NO:18, at least 6 contiguous amino acids of SEQ ID NO:20, at least 7 contiguous amino acids of SEQ ID NO:22, at least 7 contiguous amino acids of SEQ ID NO:24, at least 11 contiguous amino acids of SEQ ID NO:26, at least 257 contiguous amino acids of SEQ ID NO:28, at least 6 contiguous amino acids selected from amino acids 1-31 of SEQ ID NO:28, at least 6 contiguous amino acids of SEQ ID NO:30, at least 117 contiguous amino acids of SEQ ID NO:32, at least 6 contiguous amino acids selected from amino acids 1-65 of SEQ ID NO:32, at least 6 contiguous amino acids of SEQ ID NO:34, at least 14 contiguous amino acids of SEQ ID NO:36, at least 19 contiguous amino acids of SEQ ID NO:38, at least 8 contiguous amino acids of SEQ ID N 0:40, at least 7 contiguous amino acids of SEQ ID NO:42, and at least 10 contiguous amino acids of SEQ ID NO:44.

10. The isolated and purified subgenomic polynucleotide of claim 9 which encodes an amino acid sequence selected from the group consisting of SEQ ID NOS:2; 4, 6, 8, 10, 12, 14, 16, 18, 20, 22, 24, 26; 28, 30, 32, 34, 36, 38, 40, 42, and 44.

11. The isolated and purified subgenomic polynucleotide of claim 10 wherein the nucleotide sequence is selected from the group consisting of SEQ ID NOS:1, 3, 5, 7, 9, 11, 13, 15, 17, 19, 21, 23, 25, 27, 31, 33, 35, 37, 39, 41, and 43.

12. An isolated and purified subgenomic polynucleotide comprising a polynucleotide segment which hybridizes to a nucleotide sequence selected from the group consisting of SEQ ID NOS:1, 3, 5, 7, 9, 11, 13, 15, 17, 19, 21, 23, 25, 27, 31, 33, 35, 37, 39, 41, and 43, and the complements thereof after washing with 0.2×SSC at 65° C., wherein the polynucleotide segment encodes a protein having an amino acid sequence selected from the group consisting of SEQ ID NOS:2, 4, 6, 8, 10, 12, 14, 16, 18, 20, 22, 24, 26, 28, 30, 32, 34, 36, 38, 40, 42, and 44.

13. An isolated and purified subgenomic polynucleotide comprising a nucleotide sequence selected from the group consisting of at least 499 contiguous nucleotides of SEQ ID NO:1, at least 1141 contiguous nucleotides of SEQ ID NO:1, at least 475 contiguous nucleotides of SEQ ID NO:3, at least 313 contiguous nucleotides selected from nucleotides 1-1001 of SEQ ID NO:3, at least 751 contiguous nucleotides of SEQ ID NO:5, at least 538 contiguous nucleotides of SEQ ID NO:5, at least I 1 contiguous nucleotides selected from nucleotides 1-946 of SEQ ID NO:5, at least 13 contiguous nucleotides selected from nucleotides 1-1039 of SEQ ID NO:5, at least 651 contiguous nucleotides of SEQ ID NO:7, at least 522 contiguous nucleotides of SEQ ID NO:7, at least 11 contiguous nucleotides selected from nucleotides 1-913 of SEQ ID NO:7, at least 484 contiguous nucleotides of SEQ ID NO:9, at least 317 contiguous nucleotides of SEQ ID NO:9, at least I 1 contiguous nucleotides selected from nucleotides 1-216 of SEQ ID NO:9, at least 1 I contiguous nucleotides selected from nucleotides 379-812 of SEQ ID NO:9, at least 183 contiguous nucleotides selected from nucleotides 1-984 of SEQ ID NO:9, at least 594 contiguous nucleotides of SEQ ID NO:11, at least 289 contiguous nucleotides of SEQ ID NO: 1.1, at least 11 contiguous nucleotides selected from nucleotides 1-585 of SEQ ID NO:11, at least I 1 contiguous nucleotides selected from nucleotides 853-1120 of SEQ ID NO:I], at least 592 contiguous nucleotides of SEQ ID NO: 13, at least 275 contiguous nucleotides of SEQ ID NO: 13, at least 11 contiguous nucleotides selected from nucleotides 1-294 of SEQ ID NO:13, at least 537 contiguous nucleotides of SEQ ID NO:15, at least 294 contiguous nucleotides selected from nucleotides 1-1889 of SEQ ID NO:15, at least 171 contiguous nucleotides selected from nucleotides 318-1766 of SEQ ID NO:15, at least 11 contiguous nucleotides selected from nucleotides 1-42 of SEQ ID NO: 15; at least 11 contiguous nucleotides selected from nucleotides 478-908 of SEQ ID NO: 15, at least 11 contiguous nucleotides selected from nucleotides 1059-1078 of SEQ ID NO:15, at least 205 contiguous nucleotides of SEQ ID NO:17, at least 440 contiguous nucleotides of SEQ ID NO:19, at least 451 contiguous nucleotides of SEQ ID NO:21, at least 11 contiguous nucleotides selected from nucleotides 1-121 of SEQ ID NO:21, at least 1 I contiguous nucleotides selected from nucleotides 474-592 of SEQ ID NO:21, at least 351 contiguous nucleotides of SEQ ID NO:23, at least 21 contiguous nucleotides selected from nucleotides 1-1943 of SEQ ID NO:23, at least 11 contiguous nucleotides selected from 1-612 of SEQ ID NO:23, at least 11 contiguous nucleotides selected from nucleotides 611-719 of SEQ ID NO:23, at least 11 contiguous nucleotides selected from nucleotides 713-830 of SEQ ID NO:23, at least I I contiguous nucleotides selected from nucleotides 830-1933 of SEQ ID NO:23, at least 492 nucleotides of SEQ ID NO:25, at least I 1 contiguous nucleotides selected from nucleotides 758-847 of SEQ ID NO:25, at least 1024 contiguous nucleotides of SEQ ID NO:27, at least 347 contiguous nucleotides of SEQ ID NO:29, at least 11 contiguous nucleotides selected from nucleotides 548-601 of SEQ ID NO:29, at least 394 contiguous nucleotides of SEQ ID NO:31, at least 1 I contiguous nucleotides selected from nucleotides 1-361 of SEQ ID NO:31, at least I I contiguous nucleotides selected from nucleotides 1083-1102 of SEQ ID NO:31, at least 492 contiguous nucleotides of SEQ ID NO:33, at least 510 contiguous nucleotides of SEQ ID NO:35, at least I 1 contiguous nucleotides selected from nucleotides 1-502 or 505-631 of SEQ ID NO:35, at least 392 contiguous nucleotides of SEQ ID NO:37, at least 11 contiguous nucleotides selected from nucleotides 1-502 of SEQ ID NO:37, at least 11 contiguous nucleotides selected from nucleotides 505-631 of SEQ ID NO:37, at least 559 contiguous nucleotides of SEQ ID NO:39, at least I I contiguous nucleotides selected from nucleotides 1-92 of SEQ ID NO:39, at least 254 contiguous nucleotides of SEQ ID NO:41 at least 1 I contiguous nucleotides selected from nucleotides 1-34 of SEQ ID NO:41 at least 1 I contiguous nucleotides selected from nucleotides 55-1 10 of SEQ ID NO:41 at least 103 contiguous nucleotides of SEQ ID NO:43, at least 11 contiguous nucleotides selected from nucleotides 1-280 of SEQ ID NO:43, at least 1 I contiguous nucleotides selected from nucleotides 270-319 of SEQ ID NO:43 at least 11 contiguous nucleotides selected from nucleotides 378-423 of SEQ ID NO:43, at least I I contiguous nucleotides selected from nucleotides 414-492 of SEQ ID NO:43, at least I 1 contiguous nucleotides selected from nucleotides 532-570 of SEQ ID NO:43, at least 1 I contiguous nucleotides selected from nucleotides 1086-1152 of SEQ ID NO:43, and the complements thereof.

14. A construct comprising the isolated and purified subgenomic polynucleotide of claim 9.

15. The construct of claim 14 further comprising a promoter which is operatively linked to the nucleotide sequence.

16. A host cell comprising the construct of claim 14.

17. The host cell of claim 16 which is a mammalian cell.

18. A process for producing a protein, comprising the steps of: growing a culture of the host cell of claim 66 in a suitable culture medium; and purifying the protein secreted from the host cell.

19. A polynucleotide array comprising at least one single-stranded polynucleotide which comprises at least 12 contiguous nucleotides of a nucleotide sequence selected from the group consisting of SEQ ID NOS:1, 3, 5, 7, 9, 11, 13, 15, 17, 19, 21, 23, 25, 27, 29, 31, 33, 35, 37, 39, 41, and 43.

20. A method of detecting differential gene expression between two biological samples, comprising the step of:
contacting a first biological sample comprising single-stranded polynucleotide molecules with a first polynucleotide array comprising at least one single-stranded polynucleotide which comprises at least 12 contiguous nucleotides of a nucleotide sequence selected from the group consisting of SEQ ID NOS:1, 3, 5, 7, 9, 11, 13, 15, 17, 19, 21, 23, 25, 27, 29, 31, 33, 35, 37, 39, 41, and 43;
contacting a second biological sample comprising single-stranded polynucleotide molecules with a second polynucleotide array, wherein the first and second polynucleotide arrays comprise identical single-stranded polynucleotides; and
detecting a first and second pattern of double-stranded polynucleotides bound to the first and second polynucleotide arrays, wherein a difference between the first and second patterns indicates a gene which is differentially expressed between the first and second biological samples.

21. The method of claim 20 wherein the first biological sample is suspected of being diseased and wherein the second biological sample is not diseased.


SEQUENCE LISTING (46 sequences)

SEQ ID NO: 1 - DNA, 1325 bp, human
gttcacgttcgcggccttctgctacatgctggcgctgctgctcactgccgcgctcatcttcttcgccatttggcacattatagcatttgatgagctgaagactgattacaagaatcctatagaccagtgtaataccctgaatccccttgtactcccagagtacctcatccacgctttcttctgtgtcatgtttctttgtgcagcagagtggcttacactgggtctcaatatgcccctcttggcatatcatatttggaggtatatgagtagaccagtgatgagtggcccaggactctatgaccctacaaccatcatgaatgcagatattctagcatattgtcagaaggaaggatggtgcaaattagctttttatcttctagcatttttttactacctatatggcatgatctatgttttggtgagctcttagaacaacacacagaagaattggtccagttaagtgcatgcaaaaagccaccaaatgaagggattctatccagcaagatcctgtccaagagtagcctgtggaatctgatcagttactttaaaaaatgactccttattttttaaatgtttccacatttttgcttgtggaaagactgttttcatatgttatactcagataaagattttaaatggtattacgtataaattaatataaaatggttacctctggtgttgacaggtttgaacttgcacttcttaaggaacagccataatcctctgaatgatgcattaattactgactgtcctagtacattggaagcttttgtttataggaacttgtagggctcattttggtttcattgaaacagtatctaattataaattagctgtagatatcaggtgcttctgatgaagtgaaaatgtatatctgactagtgggaaacttcatgggtttcctcatctgtcatgtcgatgattatatatggatacatttacaaaaataaaaagcgggaattttcccttcgcttgaatattatccctgtatattgcatgaatgagagatttcccatatttccatcagagtaataaatatacttgctttaattcttaagcataagtaaacatgatataaaaatatatgctgaattacttgtgaagaatgcatttaaagctattttaaatgtgtttttatttgtaagacattacttattaagaaattggttattatgcttactgttctaatctggtggtaaaggtattcttaagaatttgcaggtactacagattttcaaaactgaatgagagaaaattgtataaccatcctgctgttcctttagtgcaatacaataaaactctgaaattaagactcaaaaaaaaaa

SEQ ID NO: 2 - protein, 142 aa, human
FTFAAFCYMLALLLTAALIFFAIWHIIAFDELKTDYKNPIDQCNTLNPLVLPEYLIHAFFCVMFLCAAEWLTLGLNMPLLAYHIWRYMSRPVMSGPGLYDPTTIMNADILAYCQKEGWCKLAFYLLAFFYYLYGMIYVLVSS

SEQ ID NO: 3 - DNA, 1277 bp, human
cacgaggaaacccacgaggggacgcggccgaggagggtcgctgtccacccgggggcgtgggagtgaggtaccagattcagcccatttggccccgacgcctctgttctcggaatccgggtgctgcggattgaggtcccggttcctaacggtgggatcggtgtcctcgggatgagatttggcgtttcctcggggctttggtgggatcggtgtcctcaggatgagatttagggtttcctcggggctttcgggatcttcacctaatatccggtattattttatgagaggagtggtcttggctgtcagaactggatccctggggtgatatttgggaattagtggagtgatctctgaagacctagggctatgatctggagctgctgtggctgaaatttggggcctctgaagtggcatggagattgaggtccagagagcctgagatcttgagggctgacatttggagagatggggtcgagggttgtctttgggccttgactgctttgggcctttctcactctcattcccgggatgctttgccagaatctctgctggattggccgtaaccctgtccccgagcgggctcacagggtctgaaggccacgcatgaggcaaaggtaaagttctgagccacccggtgcctccttcccaggactgcaagatggaggaaggcgggaacctaggaggcctgattaagatggtccatctactggtcttgtcaggtgcctggggcatgcaaatgtgggtgaccttcgtctcaggcttcctgcttttccgaagccttccccgacataccttcggactagtgcagagcaaactcttccccttctacttccacatctccatgggctgtgccttcatcaacctctgcatcttggcttcacagcatgcttgggctcagctcacattctgggaggccagccagctttacctgctgttcctgagccttacgctggccactgtcaacgcccgctggctggaaccccgcaccacagctgccatgtgggccctgcaaaccgtggagaaggagcgaggcctgggtggggaggtaccaggcagccaccagggtcccgatccctaccgccagctgcgagagaaggaccccaagtacagtgctctccgccagaatttcttccgctaccatgggctgtcctctctttgcaatctgggctgcgtcctgagcaatgggctctgtctcgctggccttgccctggaaataaggagcctctagcatgggccctgcatgctaataaatgcttcttcagaaaaaaaaaaaaaaaaaa

SEQ ID NO: 4 - protein, 189 aa, human
MEEGGNLGGLIKMVHLLVLSGAWGMQMWVTFVSGFLLFRSLPRHTFGLVQSKLFPFYFHISMGCAFINLCILASQHAWAQLTFWEASQLYLLFLSLTLATVNARWLEPRTTAAMWALQTVEKERGLGGEVPGSHQGPDPYRQLREKDPKYSALRQNFFRYHGLSSLCNLGCVLSNGLCLAGLALEIRSL

SEQ ID NO: 5 - DNA, 1610 bp, human
cacagtaggtccctcggctcagtcggcccagcccctctcagtcctccccaacccccacaaccgcccgcggctctgagacgcggccccggcggcggcggcagcagctgcagcatcatctccaccctccagccatggaagacctggaccagtctcctctggtctcgtcctcggacagcccaccccggccgcagcccgcgttcaagtaccagttcgtgagggagcccgaggacgaggaggaagaagaggaggaggaagaggaggacgaggacgaagacctggaggagctggaggtgctggagaggaagcccgccgccgggctgtccgcggccccagtgcccaccgcccctgccgccggcgcgcccctgatggacttcggaaatgacttcgtgccgccggcgccccggggacccctgccggccgctccccccgtcgccccggagcggcagccgtcttgggacccgagcccggtgtcgtcgaccgtgcccgcgccatccccgctgtctgctgccgcagtctcgccctccaagctccctgaggacgacgagcctccggcccggcctccccctcctcccccggccagcgtgagcccccaggcagagcccgtgtggaccccgccagccccggctcccgccgcgcccccctccaccccggccgcgcccaagcgcaggggctcctcgggctcagtggttgttgacctcctgtactggagagacattaagaagactggagtggtgtttggtgccagcctattcctgctgctttcattgacagtattcagcattgtgagcgtaacagcctacattgccttggccctgctctctgtgaccatcagctttaggatatacaagggtgtgatccaagctatccagaaatcagatgaaggccacccattcagggcatatctggaatctgaagttgctatatctgaggagttggttcagaagtacagtaattctgctcttggtcatgtcaactgcacgataaaggaactcaggcgcctcttcttagttgatgatttagttgattctctgaagtttgcagtgttgatgtgggtatttacctatgttggtgccttgtttaatggtctgacactactgattttggctctcatttcactcttcagtgttcctgttatttatgaacggcatcaggcacagatagatcattatctaggacttgcaaataagaatgttaaagatgctatggctaaaatccaagcaaaaatccctggattgaagcgcaaagctgaatgaaaacgcccaaaataattagtaggagttcatctttaaaggggatattcatttgattatacgggggagggtcagggaagaacgaaccttgacgttgcagtgcagtttcacagatcgttgttagatctttatttttagccatgcactgttgtgaggaaaaattacctgtcttgactgccatgtgttcatcatcttaagtattgtaagctgctatgtatggatttaaaccgtaatcatatctttttcctatctgaggcactggtggaataaaaaacctgtatattttactttgttgcagatagtcttgccgcatcttggcaagttgcagagatggtggagctagaaaaaaaaaaaaaaaaa

SEQ ID NO: 6 - protein, 373 aa, human
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 7 - DNA, 1499 bp, human
gtcgagaggacgaggtgccgctgcctggagaatcctccgctgccgtcggctcccggagcccagccctttcctaacccaacccaacctagcccagtcccagccgccagcgcctgtccctgtcacggaccccagcgttaccatgcatcctgccgtcttcctatccttacccgacctcagatgctcccttctgctcctggtaacttgggtttttactcctgtaacaactgaaataacaagtcttgatacagagaatatagatgaaattttaaacaatgctgatgttgctttagtaaatttttatgctgactggtgtcgtttcagtcagatgttgcatccaatttttgaggaagcttccgatgtcattaaggaagaatttccaaatgaaaatcaagtagtgtttgccagagttgattgtgatcagcactctgacatagcccagagatacaggataagcaaatacccaaccctcaaattgtttcgtaatgggatgatgatgaagagagaatacaggggtcagcgatcagtgaaagcattggcagattacatcaggcaacaaaaaagtgaccccattcaagaaattcgggacttagcagaaatcaccactcttgatcgcagcaaaagaaatatcattggatattttgagcaaaaggactcggacaactatagagtttttgaacgagtagcgaatattttgcatgatgactgtgcctttctttctgcatttggggatgtttcaaaaccggaaagatatagtggcgacaacataatctacaaaccaccagggcattctgctccggatatggtgtacttgggagctatgacaaattttgatgtgacttacaattggattcaagataaatgtgttcctcttgtccgagaaataacatttgaaaatggagaggaattgacagaagaaggactgccttttctcatactctttcacatgaaagaagatacagaaagtttagaaatattccagaatgaagtagctcggcaattaataagtgaaaaaggtacaataaactttttacatgccgattgtgacaaatttagacatcctcttctgcacatacagaaaactccagcagattgtcctgtaatcgctattgacagctttaggcatatgtatgtgtttggagacttcaaagatgtattaattcctggaaaactcaagcaattcgtatttgacttacattctggaaaactgcacagagaattccatcatggacctgacccaactgatacagccccaggagagcaagcccaagatgtagcaagcagtccacctgagagctccttccagaaactagcacccagtgaatataggtatactctattgagggatcgagatgagctttaaaaacttgaaaaacagtttgtaagcctttcaacagcagcatcaacctacgtggtggaaatagtaaacctatattttcataattctatgtgtatttttattttgaataaacagaaagaaatttaaaaaaaaaaaaaaaaaa

SEQ ID NO: 8 - protein, 406 aa, human
MHPAVFLSLPDLRCSLLLLVTWVFTPVTTEITSLDTENIDEILNNADVALVNFYADWCRFSQMLHPIFEEASDVIKEEFPNENQVVFARVDCDQHSDIAQRYRISKYPTLKLFRNGMMMKREYRGQRSVKALADYIRQQKSDPIQEIRDLAEITTLDRSKRNIIGYFEQKDSDNYRVFERVANILHDDCAFLSAFGDVSKPERYSGDNIIYKPPGHSAPDMVYLGAMTNFDVTYNWIQDKCVPLVREITFENGEELTEEGLPFLILFHMKEDTESLEIFQNEVARQLISEKGTINFLHADCDKFRHPLLHIQKTPADCPVIAIDSFRHMYVFGDFKDVLIPGKLKQFVFDLHSGKLHREFHHGPDPTDTAPGEQAQDVASSPPESSFQKLAPSEYRYTLLRDRDEL

SEQ ID NO: 9 - DNA, 1272 bp, human
gcctttcgcgcttctgccgtggccctctgcgggccgctccgccggtgctgtccctgggcgcctccgtgctctcagccaaccgcctctgagagcgcccactcgagcgccccgggagccagagggcgggggtcctcgccgggaccctcctgtgggcccagggggacaaaagtggctctcaatccagcacatgcacattgaagcaagttaaaggatttaatatgaagcacagaagcagatagtgccaaatagcaagcagtagttgttacacatttggtgagcagggcagcatttccttctcccactgctgctgagatggcagaaattagtcgaattcagtacgaaatggaatatactgaaggcattagtcagcgaatgagggtcccagaaaagttaaaagtagcaccgccaaacgctgacctggaacaaggattccaagaaggagttccaaatgctagtgtgataatgcaagttccggagaggattgttgtagcaggaaataatgaagatgtttcattttcaagaccagcagatcttgaccttattcagtcaactccctttaaacccctggcactgaaaacaccacctcgtgtacttacgctgagtgaaagaccactagattttctggatttagaaagacctcctacaacccctcaaaatgaagaaatccgagcagttggcagactaaaaagagagcggtctatgagtgaaaatgctgttcgccaaaatggacagctggtcagaaatgattctcttgtgacaccatcgccacaacaggctcgggtctgtcctccccatatgttacctgaagatggagctaatctttcctctgctcgtggcattttgtcgcttatccagtcttctactcgtagggcataccagcagatcttggatgtgctggatgaaaatcgcagacctgtgttgcgtggtgggtctgctgccgccacttctaatcctcatcatgacaacgtcaggtatggcatttcaaatatagatacaaccattgaaggaacgtcagatgacctgactgttgtagatgcagcttcactaagacgacagataatcaaactaaatagacgtctacaacttctggaagaggagaacaaagaacgtgctaaaagagaaatggtcatgtattcaattactgtagctttctggctgcttaatagctggctctggtttcgccgctagaggtaacatcagccctcaaaaatactgtctcaacagctggaaatataaaagatttgcaaacttcaaaaaaaaaaaaaaaaaaaa

SEQ ID NO: 10 - protein, 291 aa, human
MAEISRIQYEMEYTEGISQRMRVPEKLKVAPPNADLEQGFQEGVPNASVIMQVPERIVVAGNNEDVSFSRPADLDLIQSTPFKPLALKTPPRVLTLSERPLDFLDLERPPTTPQNEEIRAVGRLKRERSMSENAVRQNGQLVRNDSLVTPSPQQARVCPPHMLPEDGANLSSARGILSLIQSSTRRAYQQILDVLDENRRPVLRGGSAAATSNPHHDNVRYGISNIDTTIEGTSDDLTVVDAASLRRQIIKLNRRLQLLEEENKERAKREMVMYSITVAFWLLNSWLWFRR

SEQ ID NO: 11 - DNA, 1585 bp, human
gcggcccgggcgggctgctcggcgcggaacagtgctcggcatggcagggattccagggctcctcttccttctcttctttctgctctgtgctgttgggcaagtgagcccttacagtgccccctggaaacccacttggcctgcataccgcctccctgtcgtcttgccccagtctaccctcaatttagccaagccagactttggagccgaagccaaattagaagtatcttcttcatgtggaccccagtgtcataagggaactccactgcccacttacgaagaggccaagcaatatctgtcttatgaaacgctctatgccaatggcagccgcacagagacgcaggtgggcatctacatcctcagcagtagtggagatggggcccaacaccgagactcagggtcttcaggaaagtctcgaaggaagcggcagatttatggctatgacagcaggttcagcatttttgggaaggacttcctgctcaactaccctttctcaacatcagtgaagttatccacgggctgcaccggcaccctggtggcagagaagcatgtcctcacagctgcccactgcatacacgatggaaaaacctatgtgaaaggaacccagaagcttcgagtgggcttcctaaagcccaagtttaaagatggtggtcgaggggccaacgactccacttcagccatgcccgagcagatgaaatttcagtggatccgggtgaaacgcacccatgtgcccaagggttggatcaagggcaatgccaatgacatcggcatggattatgattatgccctcctggaactcaaaaagccccacaagagaaaatttatgaagattggggtgagccctcctgctaagcagctgccagggggcagaattcacttctctggttatgacaatgaccgaccaggcaatttggtgtatcgcttctgtgacgtcaaagacgagacctatgacttgctctaccagcaatgcgatgcccagccaggggccagcgggtctggggtctatgtgaggatgtggaagagacagcagcagaagtgggagcgaaaaattattggcattttttcagggcaccagtgggtggacatgaatggttccccacaggatttcaacgtggctgtcagaatcactcctctcaaatatgcccagatttgctattggattaaaggaaactacctggattgtagggaggggtgacacagtgttccctcctggcagcaattaagggtcttcatgttcttattttaggagaggccaaattgttttttgtcattggcgtgcacacgtgtgtgtgtgtgtgtgtgtgtgtgtgtgtgtaaggtgtcttataatcttttacctatttcttacaattgcaagatgactggctttactatttgaaaactggtttgtgtatcatatcatatatcatttaagcagtttgaaggcatacttttgcatagaaataaaaaaaatactgatttggggcaatgaggaatatttgacaattaagttaatcttcacgtttttgcaaactttgatttttatttcatctgaacttgtttcaaagatttatattaaatatttggcatacaagagaaaaaaaaaaaa

SEQ ID NO: 12 - protein, 383 aa, human
MAGIPGLLFLLFFLLCAVGQVSPYSAPWKPTWPAYRLPVVLPQSTLNLAKPDFGAEAKLEVSSSCGPQCHKGTPLPTYEEAKQYLSYETLYANGSRTETQVGIYILSSSGDGAQHRDSGSSGKSRRKRQIYGYDSRFSIFGKDFLLNYPFSTSVKLSTGCTGTLVAEKHVLTAAHCIHDGKTYVKGTQKLRVGFLKPKFKDGGRGANDSTSAMPEQMKFQWIRVKRTHVPKGWIKGNANDIGMDYDYALLELKKPHKRKFMKIGVSPPAKQLPGGRIHFSGYDNDRPGNLVYRFCDVKDETYDLLYQQCDAQPGASGSGVYVRMWKRQQQKWERKIIGIFSGHQWVDMNGSPQDFNVAVRITPLKYAQICYWIKGNYLDCREG

SEQ ID NO: 13 - DNA, 1071 bp, human
cagtaagctcggctcacagtcgcaggagagttctggggtacacgggcaaaggggcttgagaaggcccggaggcgaagccgaagagaagcaactgtgccccggagaagagaagctcgcccattccagactgggaaccagctttcagtgaagatggcagggccagaactgttgctcgactccaacatccgcctctgggtggtcctacccatcgttatcatcactttcttcgtaggcatgatccgccactacgtgtccatcctgctgcagagcgacaagaagctcacccaggaacaagtatctgacagtcaagtcctaattcgaagcagagtcctcagggaaaatggaaaatacattcccaaacagtctttcttgacacgaaaatattatttcaacaacccagaggatggatttttcaaaaaaactaaacggaaggtagtgccaccttctcctatgactgatcctactatgttgacagacatgatgaaagggaatgtaacaaatgtcctccctatgattcttattggtggatggatcaacatgacattctcaggctttgtcacaaccaaggtcccatttccactgaccctccgttttaagcctatgttacagcaaggaatcgagctactcacattagatgcatcctgggtgagttctgcatcctggtacttcctcaatgtatttgggcttcggagcatttactctctgattctgggccaagataatgccgctgaccaatcacgaatgatgcaggagcagatgacgggagcagccatggccatgcccgcagacacaaacaaagctttcaagacagagtgggaagctttggagctgacggatcaccagtgggcactagatgatgtcgaagaagagctcatggccaaagacctccacttcgaaggcatgttcaaaaaggaattacagacctctattttttgaagaccgagcagggattagctgtgtcaggaacttggagttgcacttaaccttgtaactttgtttggagctggcacctcttgaaataaaaaggaggatgcacgagctggcaggcatgcaaaaaaaaaaaaaaaaaaa

SEQ ID NO: 14 - protein, 261 aa, human
MAGPELLLDSNIRLWVVLPIVIITFFVGMIRHYVSILLQSDKKLTQEQVSDSQVLIRSRVLRENGKYIPKQSFLTRKYYFNNPEDGFFKKTKRKVVPPSPMTDPTMLTDMMKGNVTNVLPMILIGGWINMTFSGFVTTKVPFPLTLRFKPMLQQGIELLTLDASWVSSASWYFLNVFGLRSIYSLILGQDNAADQSRMMQEQMTGAAMAMPADTNKAFKTEWEALELTDHQWALDDVEEELMAKDLHFEGMFKKELQTSIF

SEQ ID NO: 15 - DNA, 2520 bp, human
atggcggccgccggggctgcggctacacacctagaggtggcccggggcaagcgcgccgccctcttcttcgctgcggtggccatcgtgctggggctaccgctctggtggaagaccacggagacctaccgggcctcgttgccttactcccagatcagtggcctgaatgcccttcagctccgcctcatggtgcctgtcactgtcgtgtttacgcgggagtcagtgcccctggacgaccaggagaagctgcccttcaccgttgtgcatgaaagagagattcctctgaaatacaaaatgaaaatcaaatgccgtttccagaaggcctatcggagggctttggaccatgaggaggaggccctgtcatcgggcagtgtgcaagaggcagaagccatgttagatgagcctcaggaacaagcggagggctccctgactgtgtacgtgatatctgaacactcctcacttcttccccaggacatgatgagctacattgggcccaagaggacagcagtggtgcgggggataatgcaccgggaggcctttaacatcattggccgccgcatagtccaggtggcccaggccatgtctttgactgaggatgtgcttgctgctgctctggctgaccaccttccagaggacaagtggagcgctgagaagaggcggcctctcaagtccagcttgggctatgagatcaccttcagtttactcaacccagaccccaagtcccatgatgtctactgggacattgagggggctgtccggcgctatgtgcaacctttcctgaatgccctcggtgccgctggcaacttctctgtggactctcagattctttactatgcaatgttgggggtgaatccccgctttgactcagcttcctccagctactatttggacatgcacagcctcccccatgtcatcaacccagtggagtcccggctgggatccagtgctgcctccttgtaccctgtgctcaactttctactctacgtgcctgagcttgcacactcaccgctgtacattcaggacaaggatggcgctccagtggccaccaatgccttccatagtccccgctggggtggcattatggtatataatgttgactccaaaacctataatgcctcagtgctgccagtgagagtcgaggtggacatggtgcgagtgatggaggtgttcctggcacagttgcggttgctctttgggattgctcagccccagctgcctccaaaatgcctgctttcagggcctacgagtgaagggctaatgacctgggagctagaccggctgctctgggctcggtcagtggagaacctggccacagccaccaccacccttacctccctggcgcagcttctgggcaagatcagcaacattgtcattaaggacgacgtggcatctgaggtgtacaaggctgtagctgccgtccagaagtcggcagaagagttggcgtctgggcacctggcatctgcctttgtcgccagccaggaagctgtgacatcctctgagcttgccttctttgacccgtcactcctccacctcctttatttccctgatgaccagaagtttgccatctacatcccactcttcctgcctatggctgtgcccatcctcctgtccctggtcaagatcttcctggagacccgcaagtcctggagaaagcctgagaagacagactgagcagggcagcacctccataggaagccttcctttctggccaaggtgggcggtgttagattgtgaggcacgtacatggggcctgccggaatgacttaaatatttgtctccagtctccactgttggctctccagcaaccaaagtacaacactccaagatgggttcatcttttcttcctttcccattcacctggctcaatcctcctccaccaccaggggcctcaaaaggcacatcatccgggtctccttatcttgtttgataaggctgctgcctgtctccctctgtggcaaggactgtttgttcttttgccccatttctcaacatagcacacttgtgcactgagaggagggagcattatgggaaagtccctgccttccacacctctctctagtccctgtgggacagccctagcccctgctgtcatgaaggggccaggcattggtcacctgtgggaccttctccctcactcccctccctcctagttggctttgtctgtcaggtgcagtctggcgggagtccaggaggcagcagctcaggacatggtgctgtgtgtgtgtgtgtgtgtgtgtgtgtgtgtgtgtgtgtcagaggttccagaaagttccagatttggaatcaaacagtcctgaattcaaatccttgtttttgcacttattgtctggagagctttggataaggtattgaatctctctgagcctcagtttttcatttgttcaaatggcactgatgatgtctcccttacaagatggttgtgaggagtaaatgtgatcagcatgtaaagtgtctggcgtgtagtaggctcttaataaacactggctgaatatgaattggaatgataaaaaaaaaaaaaaaaaa

SEQ ID NO: 16 - protein, 555 aa, human
MAAAGAAATHLEVARGKRAALFFAAVAIVLGLPLWWKTTETYRASLPYSQISGLNALQLRLMVPVTVVFTRESVPLDDQEKLPFTVVHEREIPLKYKMKIKCRFQKAYRRALDHEEEALSSGSVQEAEAMLDEPQEQAEGSLTVYVISEHSSLLPQDMMSYIGPKRTAVVRGIMHREAFNIIGRRIVQVAQAMSLTEDVLAAALADHLPEDKWSAEKRRPLKSSLGYEITFSLLNPDPKSHDVYWDIEGAVRRYVQPFLNALGAAGNFSVDSQILYYAMLGVNPRFDSASSSYYLDMHSLPHVINPVESRLGSSAASLYPVLNFLLYVPELAHSPLYIQDKDGAPVATNAFHSPRWGGIMVYNVDSKTYNASVLPVRVEVDMVRVMEVFLAQLRLLFGIAQPQLPPKCLLSGPTSEGLMTWELDRLLWARSVENLATATTTLTSLAQLLGKISNIVIKDDVASEVYKAVAAVQKSAEELASGHLASAFVASQEAVTSSELAFFDPSLLHLLYFPDDQKFAIYIPLFLPMAVPILLSLVKIFLETRKSWRKPEKTD

SEQ ID NO: 17 - DNA, 1245 bp, human
ctacatcctggacaacgagaccaacttcgtggtccaggtcagcgtcttcattggggtcctcatcgacctctggaagatcaccaaggtcatggacgtccggctggaccgagagcacagggtggcaggaatcttcccccgcctatccttcaaggacaagtccacgtatatcgagtcctcgaccaaagtgtatgatgatatggcattccggtacctgtcctggatcctcttcccgctcctgggctgctatgccgtctacagtcttctgtacctggagcacaagggctggtactcctgggtgctcagcatgctctacggcttcctgctgaccttcggcttcatcaccatgacgccccagctcttcatcaactacaagctcaagtctgtggcccaccttccctggcgcatgctcacctacaaggccctcaacacattcatcgacgacctgttcgcctttgtcatcaagatgcccgttatgtaccggatcggctgcctgcgggacgatgtggttttcttcatctacctctaccaacggtggatctaccgcgtcgaccccacccgagtcaacgagtttggcatgagtggagaagaccccacagctgccgcccccgtggccgaggttcccacagcagcaggggccctcacgcccacacctgcacccaccacgaccaccgccaccagggaggaggcctccacgtccctgcccaccaagcccacccagggggccagctctgccagcgagccccaggaagcccctccaaagccagcagaggacaagaaaaaggattagtcgagactggtcctcacctgctccggctcctggcgaccactacccctgcgtcccggccccctcgcctcccctccctgtcgccctttccctggacagatcaggccggggcggtgggaggcccgcctcaggtcagggcccagcgtgtgatgtaggggccggggcaggccagggtttgtttgtggaggcgctgtctgtccctctgtccctctgtgtttccagccatctcgccctgccagcccagcaccactgggaatcatggtgaagctgatgcagcgttgccgagggggtgggttgggcgggggtggggccgggcccccctaggggatgccccgggccgttcatcatcttgtccctggtccccctaccacactccccctcctaaaccgccgccctttaacacagtttggatttaataaattcagatgggggtttaacttaaactcaaaaaaaaaaaaaaa

SEQ ID NO: 18 - protein, 232 aa, human
MDVRLDREHRVAGIFPRLSFKDKSTYIESSTKVYDDMAFRYLSWILFPLLGCYAVYSLLYLEHKGWYSWVLSMLYGFLLTFGFITMTPQLFINYKLKSVAHLPWRMLTYKALNTFIDDLFAFVIKMPVMYRIGCLRDDVVFFIYLYQRWIYRVDPTRVNEFGMSGEDPTAAAPVAEVPTAAGALTPTPAPTTTTATREEASTSLPTKPTQGASSASEPQEAPPKPAEDKKKD

SEQ ID NO: 19 - DNA, 1030 bp, human
aacatggagactttgtaccgtgtcccgttcttagtgctcgaatgtcccaacctgaagctgaagaagccgccctggttgcacatgccgtcggccatgactgtgtatgctctggtggtggtgtcttacttcctcatcaccggaggaataatttatgatgttattgttgaacctccaagtgtcggttctatgactgatgaacatgggcatcagaggccagtagctttcttggcctacagagtaaatggacaatatattatggaaggacttgcatccagcttcctatttacaatgggaggtttaggtttcataatcctggaccgatcgaatgcaccaaatatcccaaaactcaatagattccttcttctgttcattggattcgtctgtgtcctattgagttttttcatggctagagtattcatgagaatgaaactgccgggctatctgatgggttagagtgcctttgagaagaaatcagtggatactggatttgctcctgtcaatgaagttttaaaggctgtaccaatcctctaatatgaaatgtggaaaagaatgaagagcagcagtaaaagaaatatctagtgaaaaaacaggaagcgtattgaagcttggactagaatttcttcttggtattaaagagacaagtttatcacagaattttttttcctgctggcctattgctataccaatgatgttgagtggcattttctttttagtttttcattaaaatatattccatatctacaactataatatcaaataaagtgattattttttacaaccctcttaacattttttggagatgacatttctgattttcagaaattaacataaaatccagaagcaagattccgtaagctgagaactctggacagttgatcagctttacctatggtgctttgcctttaactagagtgtgtgatggtagattatttcagatatgtatgtaaaactgtttcctgaacaataagatgtatgaacggagcagaaataaatactttttctaattaatacctttaaaaaaaaaaaaaaa

SEQ ID NO: 20 - protein, 149 aa, human
METLYRVPFLVLECPNLKLKKPPWLHMPSAMTVYALVVVSYFLITGGIIYDVIVEPPSVGSMTDEHGHQRPVAFLAYRVNGQYIMEGLASSFLFTMGGLGFIILDRSNAPNIPKLNRFLLLFIGFVCVLLSFFMARVFMRMKLPGYLMG

SEQ ID NO: 21 - DNA, 1563 bp, human
gttgattgggtctagaccaaagaactttgaggaacttgcccagagccctgcatgcatcagacctacagcagacattgcaggcctgaagaaaggtggtcacaagaggggtggaacattcctgcaaatggtttcaatatatgcagatgtctcgatataggaatgaaattacgtctttggaacaacttaaataagtcaaatatacttggagctttaaaaattaaaaggagagagattcgagcaccttttctgctgccatgacaaccatgcaaggaatggaacaggccatgccaggggctggccctggtgtgccccagctgggaaacatggctgtcatacattcacatctgtggaaaggattgcaagagaagttcttgaagggagaacccaaagtccttggggttgtgcagattctgactgccctgatgagccttagcatgggaataacaatgatgtgtatggcatctaatacttatggaagtaaccctatttccgtgtatatcgggtacacaatttgggggtcagtaatgtttattatttcaggatccttgtcaattgcagcaggaattagaactacaaaaggcctgggtctggatggcatggtgctcctcttaagtgtgctggaattctgcattgctgtgtccctctctgcctttggatgtaaagtgctctgttgtacccctggtggggttgtgttaattctgccatcacattctcacatggcagaaacagcatctcccacaccacttaatgaggtttgaggccaccaaaagatcaacagacaaatgctccagaaatctatgctgactgtgacacaagagcctcacatgagaaattaccagtatccaacttcgatactgatagacttgttgatattattattatatgtaatccaattatgaactgtgtgtgtatagagagataataaattcaaaattatgttctcatttttttccctggaactcaataactcatttcactggctctttatcgagagtactagaagttaaattaataaataatgcatttaatgaggcaacagcacttgaaagtttttcattcatcataagaactttatataaaggcattacattggcaaataaggtttggaagcagaagagcaaaaaaaagatattgttaaaatgaggcctccatgcaaaacacatacttccctcccatttatttaacttttttttttctcctacctatggggaccaaagtgctttttccttcaggaagtggagatgcatggccatctccccctccctttttccttctcctgcttttctttccccatagaaagtaccttgaagtagcacagtccgtccttgcatgtgcacgagctatcatttgagtaaaagtatacatggagtaaaaatcatattaagcatcagattcaacttatattttctatttcatcttcttcctttcccttctcccaccttctactgggcataattatatcttaatcatatatggaaatgtgcaacatatggtatttgttaaatacgtttgtttttattgcagagcaaaaataaatcaaattagaagcaaaaaaaaaaaaaa

SEQ ID NO: 22 - protein, 167 aa, human
MTTMQGMEQAMPGAGPGVPQLGNMAVIHSHLWKGLQEKFLKGEPKVLGVVQILTALMSLSMGITMMCMASNTYGSNPISVYIGYTIWGSVMFIISGSLSIAAGIRTTKGLGLDGMVLLLSVLEFCIAVSLSAFGCKVLCCTPGGVVLILPSHSHMAETASPTPLNEV

SEQ ID NO: 23 - DNA, 2590 bp, human
ccggcgggacggagggcccggcaggaagatgggctcccgtggacagggactcttgctggcgtactgcctgctccttgcctttgcctctggcctggtcctgagtcgtgtgccccatgtccagggggaacagcaggagtgggaggggactgaggagctgccgtcgcctccggaccatgccgagagggctgaagaacaacatgaaaaatacaggcccagtcaggaccaggggctccctgcttcccggtgcttgcgctgctgtgaccccggtacctccatgtacccggcgaccgccgtgccccagatcaacatcactatcttgaaaggggagaagggtgaccgcggagatcgaggcctccaagggaaatatggcaaaacaggctcagcaggggccaggggccacactggacccaaagggcagaagggctccatgggggcccctggggagcggtgcaagagccactacgccgccttttcggtgggccggaagaagcccatgcacagcaaccactactaccagacggtgatcttcgacacggagttcgtgaacctctacgaccacttcaacatgttcaccggcaagttctactgctacgtgcccggcctctacttcttcagcctcaacgtgcacacctggaaccagaaggagacctacctgcacatcatgaagaacgaggaggaggtggtgatcttgttcgcgcaggtgggcgaccgcagcatcatgcaaagccagagcctgatgctggagctgcgagagcaggaccaggtgtgggtacgcctctacaagggcgaacgtgagaacgccatcttcagcgaggagctggacacctacatcaccttcagtggctacctggtcaagcacgccaccgagccctagctggccggccacctcctttcctctcgccaccttccacccctgcgctgtgctgaccccaccgcctcttccccgatccctggactccgactccctggctttggcattcagtgagacgccctgcacacacagaaagccaaagcgatcggtgctcccagatcccgcagcctctggagagagctgacggcagatgaaatcaccagggcggggcacccgcgagaaccctctgggaccttccgcggccctctctgcacacatcctcaagtgaccccgcacggcgagacgcgggtggcggcagggcgtcccagggtgcggcaccgcggctccagtccttggaaataattaggcaaattctaaaggtctcaaaaggagcaaagtaaaccgtggaggacaaagaaaagggttgttatttttgtctttccagccagcctgctggctcccaagagagaggccttttcagttgagactctgcttaagagaagatccaaagttaaagctctggggtcaggggaggggccgggggcaggaaactacctctggcttaattcttttaagccacgtaggaactttcttgagggataggtggaccctgacatccctgtggccttgcccaagggctctgctggtctttctgagtcacagctgcgaggtgatgggggctggggccccaggcgtcagcctcccagagggacagctgagccccctgccttggctccaggttggtagaagcagccgaagggctcctgacagtggccagggacccctgggtcccccaggcctgcagatgtttctatgaggggcagagctcctggtacatccatgtgtggctctgctccacccctgtgccaccccagagccctggggggtggtctccatgcctgccaccctggcatcggctttctgtgccgcctcccacacaaatcagccccagaaggccccggggccttggcttctgttttttataaaacacctcaagcagcactgcagtctcccatctcctcgtgggctaagcatcaccgcttccacgtgtgttgtgttggttggcagcaaggctgatccagaccccttctgcccccactgccctcatccaggcctctgaccagtagcctgagaggggctttttctaggcttcagagcaggggagagctggaaggggctagaaagctcccgcttgtctgtttctcaggctcctgtgagcctcagtcctgagaccagagtcaagaggaagtacacgtcccaatcacccgtgtcaggattcactctcaggagctgggtggcaggagaggcaatagcccctgtggcaattgcaggaccagctggagcagggttgcggtgtctccacggtgctctcgccctgcccatggccaccccagactctgatctccaggaaccccatagcccctctccacctcaccccatgttgatgcccagggtcactcttgctacccgctgggcccccaaacccccgctgcctctcttccttccccccatcccccacctggttttgactaatcctgcttccctctctgggcctggctgccgggatctggggtccctaagtccctctctttaaagaacttctgcgggtcagactctgaagccgagttgctgtgggcgtgcccggaagcagagcgccacactcgctgcttaagctcccccagctctttccagaaaacattaaactcagaattgtgttttcaaaaaaaaaaaaaaaaaaaa

SEQ ID NO: 24 - protein, 281 aa, human
MGSRGQGLLLAYCLLLAFASGLVLSRVPHVQGEQQEWEGTEELPSPPDHAERAEEQHEKYRPSQDQGLPASRCLRCCDPGTSMYPATAVPQINITILKGEKGDRGDRGLQGKYGKTGSAGARGHTGPKGQKGSMGAPGERCKSHYAAFSVGRKKPMHSNHYYQTVIFDTEFVNLYDHFNMFTGKFYCYVPGLYFFSLNVHTWNQKETYLHIMKNEEEVVILFAQVGDRSIMQSQSLMLELREQDQVWVRLYKGERENAIFSEELDTYITFSGYLVKHATEP

SEQ ID NO: 25 - DNA, 1668 bp, human
ggacttgagcgagccagttgccggattattctatttcccctccctctctcccgccccgtatctcttttcacccttctcccaccctcgctcgcgtagccatggcggagccgtcggcggccactcagtcccattccatctcctcgtcgtccttcggagccgagccgtccgcgcccggcggcggcgggagcccaggagcctgccccgccctggggacgaagagctgcagctcctcctgtgcggtgcacgatctgattttctggagagatgtgaagaagactgggtttgtctttggcaccacgctgatcatgctgctttccctggcagctttcagtgtcatcagtgtggtttcttacctcatcctggctcttctctctgtcaccatcagcttcaggatctacaagtccgtcatccaagctgtacagaagtcagaagaaggccatccattcaaagcctacctggacgtagacattactctgtcctcagaagctttccataattacatgaatgctgccatggtgcacatcaacagggccctgaaactcattattcgtctctttctggtagaagatctggttgactccttgaagctggctgtcttcatgtggctgatgacctatgttggtgctgtttttaacggaatcacccttctaattcttgctgaactgctcattttcagtgtcccgattgtctatgagaagtacaagacccagattgatcactatgttggcatcgcccgagatcagaccaagtcaattgttgaaaagatccaagcaaaactccctggaatcgccaaaaaaaaggcagaataagtacatggaaaccagaaatgcaacagttactaaaacaccatttaatagttataacgtcgttacttgtactatgaaggaaaatactcagtgtcagcttgagcctgcattccaagctttttttttaatttggtgttttctcccatcctttccctttaaccctcagtatcaagcacaaaaattgatggactgataaaagaactatcttagaactcagaagaagaaagaatcaaattcataggataagtcaataccttaatggtggtagagcctttacctgtagcttgaaaggggaaagattggaggtaagagagaaaatgaaagaacacctctgggtccttctgtccagttttcagcactagtcttactcagctatccattatagttttgcccttaagaagtcatgattaacttatgaaaaaattatttggggacaggagtgtgataccttccttggtttttttttgcagccctcaaatcctatcttcctgccccacaatgtgagcagctacccctgatactccttttctttaatgatttaactatcaacttgataaataacttataggtgatagtgataattcctgattccaagaatgccatctgataaaaaagaatagaaatggaaagtgggactgagagggagtcagcaggcatgctgcggtggcggtcactccctctgccactatccccagggaaggaaaggctccgccatttgggaaagtggtttctacgtcactggacaccggttctgagcattagtttgagaactcgttcccgaatgtgctttcctccctctcccctgcccacctcaagtttaataaataaggttgtacttttcttactataaaaaaaaaaaaaa

SEQ ID NO: 26 - protein, 236 aa, human
MAEPSAATQSHSISSSSFGAEPSAPGGGGSPGACPALGTKSCSSSCAVHDLIFWRDVKKTGFVFGTTLIMLLSLAAFSVISVVSYLILALLSVTISFRIYKSVIQAVQKSEEGHPFKAYLDVDITLSSEAFHNYMNAAMVHINRALKLIIRLFLVEDLVDSLKLAVFMWLMTYVGAVFNGITLLILAELLIFSVPIVYEKYKTQIDHYVGIARDQTKSIVEKIQAKLPGIAKKKAE

SEQ ID NO: 27 - DNA, 1697 bp, human
cttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccgctgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacgtcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccccaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaaaaaaaaaaaaaaa

SEQ ID NO: 28 - protein, 466 aa, human
MFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDVVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSPETMIKWTREKE

SEQ ID NO: 29 - DNA, 1333 bp, human
ggtgggtgcatcctgcgctgcggcgggcgcgctacccagacgctggtgtgcagagccacatgaagcctgctggggactgggggccagggagcagcaagccagctgggactgaggcggacgctgtctcagggagacgctgactcgcaaagacactcccttccttgtgcctgggtaaaaagtctcctcctggggtccctggccatcctgaatatccagaatggtgtttctgaagttcttctgcatgagtttcttctgccacctgtgtcaaggctacttcgatggccccctctacccagagatgtccaatgggactctgcaccactacttcgtgcccgatggggactatgaggagaacgatgaccccgagaagtgccagctgctcttcagggtgagtgaccacaggcgctgctcccagggggaggggagccaggttggcagcctgctgagcctcaccctgcgggaggagttcaccgtgctgggccgccaggtggaggatgctgggcgcgtgctggagggcatcagcaaaagcatctcctacgacctagacggggaagagagctatggcaagtacctgcggcgggagtcccaccagatcggggatgcctactccaactcggacaaatccctcactgagctggagagcaagttcaagcagggccaggaacaggacagccggcaggagagcaggctcaacgaggactttctgggaatgctggtccacaccaggtccctgctgaaggagacactggacatctctgtggggctcagggacaaatacgagctgctggccctcaccattaggagccatgggacccgactaggtcggctgaaaaatgattatcttaaagtataggtggaaggatacaaatgctagaaagagggaatcaaatcagccccgttttggagggtgggggacagaagatggggctacatttcccccatacctactatttttttatatcccgatttgcactttgagaatacatctaaggtcatctttcaaaagagaaaaattggacacttgagtgactttgtttttagttttgtttttgtacattatttatgtgattgttatggaattgtcacctggaaagaacaattttaagcaatgtcatttctagatgggtttctaattctgcagagacacccgtttcagccacatctaaaagagcacagtttatgtggtgcggaattaaacttccccatcctgcagattatgtggaaatacccaaagataatagtgcatagctcctttcagcctctagccttcactcctgggctccaaaagctatcccagttgcctgtttttcaaatgaggttcaaggtgctgctt

SEQ ID NO: 30 - protein, 211 aa, human
MVFLKFFCMSFFCHLCQGYFDGPLYPEMSNGTLHHYFVPDGDYEENDDPEKCQLLFRVSDHRRCSQGEGSQVGSLLSLTLREEFTVLGRQVEDAGRVLEGISKSISYDLDGEESYGKYLRRESHQIGDAYSNSDKSLTELESKFKQGQEQDSRQESRLNEDFLGMLVHTRSLLKETLDISVGLRDKYELLALTIRSHGTRLGRLKNDYLKV

SEQ ID NO: 31 - DNA, 1102 bp, human
gtcttggggtccctggctgggtggccagaccccgaagccagcgctgggaagggctgcggatgcccgggtcagaggaaggggcaggtccaaggacacgcgggtctggtcctgggcaagaaccgccccctctccgggcctgcttcagtcttcctttgcagaacaacgggccaggccccttccctctgcccccgggtgcttgaagtctagccccatcctggtccaatgcgctcttggtagcctcctttcccagctgcccgcccgccgccatgccgcccttactgcccctgcgcctgtgccggctgtggccccgcaaccctccctcccggctcctcggagcggccgccgggcagcggtccagacccagtacttattatgaactgttgggggtgcatcctggtgccagcactgaggaagttaaacgagctttcttctccaagtccaaagagctgcacccagaccgggaccctgggaacccaagcctgcacagccgctttgtggagctgagcgaggcataccgtgtgctcagccgtgagcagagccgccgcagctatgatgaccagctccgctcaggtagtcccccaaagtctccacgaaccacagtccatgacaagtctgcccaccaaacacacagctcctggacaccccccaacgcacagtactggtcccagtttcacagcgtgaggccacaggggccccagttgaggcagcagcaacacaaacaaaacaaacaagtgctggggtactgcctcctcctcatgctggcgggcatgggcctgcactacattgccttcaggaaggtgaagcagatgcaccttaacttcatggatgaaaaggatcggatcatcacagccttctacaacgaagcccgggcacgggccagggccaacagaggcatccttcagcaggagcgacaacggctagggcagcggcagccgccaccatccgagccaacccaaggccccgagatcgtgccccggggcgccggcccctgaggggctcacctggatggggcctgcagtgcgttcccgctttgcttccttccctggacggcccgctccccgaaacgcgcgcaataaagtgattcgcagaaaaaaaaaaaaaa

SEQ ID NO: 32 - protein, 241 aa, human
MPPLLPLRLCRLWPRNPPSRLLGAAAGQRSRPSTYYELLGVHPGASTEEVKRAFFSKSKELHPDRDPGNPSLHSRFVELSEAYRVLSREQSRRSYDDQLRSGSPPKSPRTTVHDKSAHQTHSSWTPPNAQYWSQFHSVRPQGPQLRQQQHKQNKQVLGYCLLLMLAGMGLHYIAFRKVKQMHLNFMDEKDRIITAFYNEARARARANRGILQQERQRLGQRQPPPSEPTQGPEIVPRGAGP

SEQ ID NO: 33 - DNA, 966 bp, human
gagaagcatcgaggctataggacgcagctgttgccatgacggcccaggggggcctggtggctaaccgaggccggcgcttcaagtgggccattgagctaagcgggcctggaggaggcagcaggggtcgaagtgaccggggcagtggccagggagactcgctctacccagtcggttacttggacaagcaagtgcctgataccagcgtgcaagagacagaccggatcctggtggagaagcgctgctgggacatcgccttgggtcccctcaaacagattcccatgaatctcttcatcatgtacatggcaggcaatactatctccatcttccctactatgatggtgtgtatgatggcctggcgacccattcaggcacttatggccatttcagccactttcaagatgttagaaagttcaagccagaagtttcttcagggtttggtctatctcattgggaacctgatgggtttggcattggctgtttacaagtgccagtccatgggactgttacctacacatgcatcggattggttagccttcattgagccccctgagagaatggagttcagtggtggaggactgcttttgtgaacatgagaaagcagcgcctggtccctatgtatttgggtcttatttacatccttctttaagcccagtggctcctcagcatactcttaaactaatcacttatgttaaaaagaaccaaaagactcttttctccatggtggggtgacaggtcctagaaggacaatgtgcatattacgacaaacacaaagaaactataccataacccaaggctgaaaataatgtagaaaactttatttttgtttccagtacagagcaaaacaacaacaaaaaaacataactatgtaaacaagagaataactgctgctaaatcaagaactgttgcagcatctcctttcaataaattaaatggttgagaacaatgcataaaaaaaaaaaaaaaaaa

SEQ ID NO: 34 - protein, 183 aa, human
MTAQGGLVANRGRRFKWAIELSGPGGGSRGRSDRGSGQGDSLYPVGYLDKQVPDTSVQETDRILVEKRCWDIALGPLKQIPMNLFIMYMAGNTISIFPTMMVCMMAWRPIQALMAISATFKMLESSSQKFLQGLVYLIGNLMGLALAVYKCQSMGLLPTHASDWLAFIEPPERMEFSGGGLLL

SEQ ID NO: 35 - DNA, 1570 bp, human
gggccgggcgcggcgcagaggcgggcgcctaccagccggcagctccggagctgcccgcgccatgtccgcgcacaatcggggcaccgagctcgaccttagctggatctccaaaatacaagtgaatcacccggcagttctgaggcgtgcggaacaaatccaggctcgcagaaccgtgaaaaaggagtggcaggctgcttggctcctgaaagctgttacctttatagatcttactacactttcaggtgatgatacatcttccaacattcaaaggctctgttataaagccaaatacccaatccgggaagatctcttaaaagctttaaatatgcatgataaaggcattactacagccgccgtttgtgtttatcccgcccgggtgtgtgatgctgtaaaagcactcaaggctgcaggctgtaatatccctgtggcatcagtggccgctggatttccagctggacagactcatttgaagacacgattagaagagatcagattggctgtggaagatggagctacagaaatcgacgtggtaattaacagaagcttggtgctgacaggccagtgggaagccctgtacgatgagattcgtcagtttcgcaaggcctgtggggaggctcatcttaaaactatattagcgacaggagaacttggaactcttactaatgtctataaagccagtatgatagcaatgatggcaggatcagattttattaagacctctactggaaaagaaacagtaaatgccaccttcccggtagctatagtaatgctgcgggccattagagatttcttctggaaaactggaaacaagatagggtttaaaccagcaggaggcatccgcagtgcaaaggattcccttgcttggctctctcttgtaaaggaggagcttggagatgagtggctgaagccagaactctttcgaataggtgccagtactctgctctcggacattgagaggcagatttaccatcatgtgactggaagatatgcagcttatcatgatcttccaatgtcttaaatcagtcaccagttccagaaaagttctttacgacaatgtttaaaaattatttttctacgtaattgctaaaattatttaattaaaaaattgggcagtaggtaactggcattcctctctttaaaatttctaccgaacttaatggaatggaaaaagcaaactcatccacatgtggtactcatttcaggcacatctgaaatgatcttaattactagaagatctgcactattaactttgtgaagagtttctcctaaaaactttaagtaaaatgttaatggtagctttgataacatcaaattctaagggagaaaaaaacaatattaaaccgcccaagcagtgtgccctagcagaggaaaatgcaacatctcgcaagcgctgctgtaacgacttcaggagtcactgattcagcactaatttcctgctgtgaaaactcatctttcatttttgccgtggataggcgcttttattaattgttgtcctaatgaaatttctgacattgtcatatacaacgatgaatatcattaaaatttttaaaataaaaaaaaaaaaaaaaaa

SEQ ID NO: 36 - protein, 318 aa, human
MSAHNRGTELDLSWISKIQVNHPAVLRRAEQIQARRTVKKEWQAAWLLKAVTFIDLTTLSGDDTSSNIQRLCYKAKYPIREDLLKALNMHDKGITTAAVCVYPARVCDAVKALKAAGCNIPVASVAAGFPAGQTHLKTRLEEIRLAVEDGATEIDVVINRSLVLTGQWEALYDEIRQFRKACGEAHLKTILATGELGTLTNVYKASMIAMMAGSDFIKTSTGKETVNATFPVAIVMLRAIRDFFWKTGNKIGFKPAGGIRSAKDSLAWLSLVKEELGDEWLKPELFRIGASTLLSDIERQIYHHVTGRYAAYHDLPMS

SEQ ID NO: 37 - DNA, 1542 bp, human
ccaacttccaactccctgtcctgtcctaggtaacccctccaccccgccattctcctatcccgtgtctgtccccatccctgtgacccctgacccctggcctttgccactccccagggaccgatgatgtggcgaccatcagttctgctgcttctgttgctactgaggcacggggcccaggggaagccatccccagacgcaggccctcatggccaggggagggtgcaccaggcggcccccctgagcgacgctccccatgatgacgcccacgggaacttccagtacgaccatgaggctttcctgggacgggaagtggccaaggaattcgaccaactcaccccagaggaaagccaggcccgtctggggcggatcgtggaccgcatggaccgcgcgggggacggcgacggctgggtgtcgctggccgagcttcgcgcgtggatcgcgcacacgcagcagcggcacatacgggactcggtgagcgcggcctgggacacgtacgacacggaccgcgacgggcgtgtgggttgggaggagctgcgcaacgccacctatggccactacgcgcccggtgaagaatttcatgacgtggaggatgcagagacctacaaaaagatgctggctcgggacgagcggcgtttccgggtggccgaccaggatggggactcgatggccactcgagaggagctgacagccttcctgcaccccgaggagttccctcacatgcgggacatcgtgattgctgaaaccctggaggacctggacagaaacaaagatggctatgtccaggtggaggagtacatcgcggatctgtactcagccgagcctggggaggaggagccggcgtgggtgcagacggagaggcagcagttccgggacttccgggatctgaacaaggatgggcacctggatgggagtgaggtgggccactgggtgctgccccctgcccaggaccagcccctggtggaagccaaccacctgctgcacgagagcgacacggacaaggatgggcggctgagcaaagcggaaatcctgggtaattggaacatgtttgtgggcagtcaggccaccaactatggcgaggacctgacccggcaccacgatgagctgtgagcaccgcgcacctgccacagcctcagaggcccgcacaatgaccggaggaggggccgctgtggtctggccccctccctgtccaggccccgcaggaggcagatgcagtcccaggcatcctcctgcccctgggctctcagggaccccctgggtcggcttctgtccctgtcacacccccaaccccagggaggggctgtcatagtcccagaggataagcaatacctatttctgactgagtctcccagcccagacccagggacccttggccccaagctcagctctaagaaccgccccaacccctccagctccaaatctgagcctccaccacatagactgaaactcccctggccccagccctctcctgcctggcctggcctgggacacctcctctctgccaggaggcaataaaagccagcgccgggaaaaaaaaaaaaaaaa

SEQ ID NO: 38 - protein, 328 aa, 38
MMWRPSVLLLLLLLRHGAQGKPSPDAGPHGQGRVHQAAPLSDAPHDDAHGNFQYDHEAFLGREVAKEFDQLTPEESQARLGRIVDRMDRAGDGDGWVSLAELRAWIAHTQQRHIRDSVSAAWDTYDTDRDGRVGWEELRNATYGHYAPGEEFHDVEDAETYKKMLARDERRFRVADQDGDSMATREELTAFLHPEEFPHMRDIVIAETLEDLDRNKDGYVQVEEYIADLYSAEPGEEEPAWVQTERQQFRDFRDLNKDGHLDGSEVGHWVLPPAQDQPLVEANHLLHESDTDKDGRLSKAEILGNWNMFVGSQATNYGEDLTRHHDEL

SEQ ID NO: 39 - DNA, 1990 bp, human
cacgagcctgcccggcccccggctccagcgagcgagcggcgagcaggcggctcacagaggcctggccgcccacggaacccggggcccggcggccgccgccgcgatgtttccccgcgagaagacgtggaacatctcgttcgcgggctgcggcttcctcggcgtctactacgtcggcgtggcctcctgcctccgcgagcacgcgcccttcctggtggccaacgccacgcacatctacggcgcctcggccggggcgctcacggccacggcgctggtcaccggggtctgcctgggtgaggctggtgccaagttcattgaggtatctaaagaggcccggaagcggttcctgggccccctgcacccctccttcaacctggtaaagatcatccgcagtttcctgctgaaggtcctgcctgctgatagccatgagcatgccagtgggcgcctgggcatctccctgacccgcgtgtcagacggcgagaatgtcattatatcccacttcaactccaaggacgagctcatccaggccaatgtctgcagcggtttcatccccgtgtactgtgggctcatccctccctccctccagggggtgcgctacgtggatggtggcatttcagacaacctgccactctatgagcttaagaacaccatcacagtgtcccccttctcgggcgagagtgacatctgtccgcaggacagctccaccaacatccacgagctgcgggtcaccaacaccagcatccagttcaacctgcgcaacctctaccgcctctccaaggccctcttcccgccggagcccctggtgctgcgagagatgtgcaagcagggataccgggatggcctgcgctttctgcagcggaacggcctcctgaaccggcccaaccccttgctggcgttgccccccgcccgcccccacggcccagaggacaaggaccaggcagtggagagcgcccaagcggaggattactcgcagctgcccggagaagatcacatcctggagcacctgcccgcccggctcaatgaggccctgctggaggcctgcgtggagcccacggacctgctgaccaccctctccaacatgctgcctgtgcgtctggccacggccatgatggtgccctacacgctgccgctggagagcgctctgtccttcaccatccgcttgctggagtggctgcccgacgttcccgaggacatccggtggatgaaggagcagacgggcagcatctgccagtacctggtgatgcgcgccaagaggaagctgggcaggcacctgccctccaggctgccggagcaggtggagctgcgccgcgtccagtcgctgccgtccgtgccgctgtcctgcgccgcctacagagaggcactgcccggctggatgcgcaacaacctctcgctgggggacgcgctggccaagtgggaggagtgccagcgccagctgctgctcggcctcttctgcaccaacgtggccttcccgcccgaagctctgcgcatgcgcgcacccgccgacccggctcccgcccccgcggacccagcatccccgcagcaccagccggccgggcctgcccccttgctgagcacccctgctcccgaggcccggcccgtgatcggggccctggggctgtgagaccccgaccctctcgaggaaccctgcctgagacgcctccattaccactgcgcagtgagatgaggggactcacagttgccaagaggggtctttgccgtgggccccctcgccagccactcaccagctgcactgagaggggaggtttccacacccctcccctgggccgctgaggccccgcgcacctgtgccttaatcttccctcccctgtgctgcccgagcacctcccccgcccctttactcctgggaactttgcagctgcccttccctccccgtttttcatggcctgctgaaatatgtgtgtgaagaattatttattttcgccaaagcacatgtaataaatgctgcagcccagaaaaaaaaaaaaaaaaaaaa

SEQ ID NO: 40 - protein, 504 aa, human [sequence omitted]

SEQ ID NO: 41 - DNA, 684 bp, human
accgtcatgctccagttctttgtgcacttcctgagccttgtctacctgtaccgtgaggcccaggcccggagccccgagaagcaggagcagttcgtggacttgtacaaggagtttgagccaagcctggtcaacagcaccgtctacatcatggccatggccatgcagatggccaccttcgccatcaattacaaaggcccgcccttcatggagagcctgcccgagaacaagcccctggtgtggagtctggcagtttcactcctggccatcattggcctgctcctcggctcctcgcccgacttcaacagccagtttggcctcgtggacatccctgtggagttcaagctggtcattgcccaggtcctgctcctggacttctgcctggcgctcctggccgaccgcgtcctgcagttcttcctggggaccccgaagctgaaagtgccttcctgagatggcagtgctggtacccactgcccaccctggctgccgctgggcgggaaccccaacagggccccgggagggaaccctgcccccaaccccccacagcaaggctgtacagtctcgcccttggaagactgagctgggacccccacagccatccgctggcttggccagcagaaccagccccaagccagcacctttggtaaataaagcagcatctgagattttaaaaaaaaaaaaaaaaaa

SEQ ID NO: 42 - protein, 146 aa, human [sequence omitted]

SEQ ID NO: 43 - DNA, 1152 bp, human
ggcacgagggcagcctcccctcgctcgctctcctcttcctctagggccccagcgcagctcgggagcccgcgcaccgaggcgctaggggcaccgcgcactagagggacacccgccgcgcctggacagcccccggcgggcgcccccctcgcacctcctgccccgcgcgggccgcgctcccctcccccgcgcctgtgtccccagggcgcagggccgcgcgtccagccccagacccgccggggtccctggggacgcgccagcccggcagtggctcgacgatggaggagccgcagcgcgcccgctcgcacacagtcaccaccaccgccagctccttcgcagagaacttctccaccagcagcagcagcttcgcctacgaccgggagttcctccgcaccctgcccggcttcctcatcgtggccgagatcgttctggggctgctggtatggacgcttattgctggaactgagtacttccgggtccccgcatttggctgggtcatgtttgtagctgtattttactgggtcctcaccgtcttcttcctcattatctacataacaatgacctacaccaggattccccaggtgccctggacaacagtgggcctgtgctttaacggcagtgccttcgtcttgtacctctctgccgctgttgtagatgcatcttccgtctcccctgagagggacagtcacaacttcaacagctgggcggcctcatcgttctttgccttcctggtcaccatctgctacgctggaaatacatatttcagttttatagcatggagatccaggaccatacagtgatttaccattttgataattaaaaggaaaaaaaaaggaagactctcactgtaaaaacagctgtaggtataatgtatattcccagagaattgtatttaactaattaatgttttttatattcttaaatttgctcacaaattgtggtttgttacaattaaactggatacttatttgcaaagtgttgtagcttataatgaactcttaagtatcttattaatgtattaatgtcttcatagatcatattttcttagacaatgtttaaatagataaattgctaatattgagaatgtgtcaagtttgtaaacctaacttttaagatgccagattcttttttgattaaatgttgcaaaatcccaaa

SEQ ID NO: 44 - protein, 173 aa, human
MEEPQRARSHTVTTTASSFAENFSTSSSSFAYDREFLRTLPGFLIVAEIVLGLLVWTLIAGTEYFRVPAFGWVMFVAVFYWVLTVFFLIIYITMTYTRIPQVPWTTVGLCFNGSAFVLYLSAAVVDASSVSPERDSHNFNSWAASSFFAFLVTICYAGNTYFSFIAWRSRTIQ

SEQ ID NO: 45 - DNA, 6 bp, human
aataaa

SEQ ID NO: 46 - DNA, 6 bp, human
attaaa